(19) United States
(12) Patent Application Publication
(10) Pub. No.: US 2026/0090985 A1
(43) Pub. Date: Apr. 2, 2026

(54) ORAL MUCOSAL FORMULATIONS OF ASPIRIN

(71) Applicant: Aspire Biopharma Holdings, Inc., Estero, FL (US)

(72) Inventor: James Dzandu, Phoenix, AZ (US)

(21) Appl. No.: 19/348,728

(22) Filed: Oct. 2, 2025

Related U.S. Application Data
(60) Provisional application No. 63/702,381, filed on Oct. 2, 2024.
(60) Provisional application No. 63/864,091, filed on Aug. 14, 2025.

(51) Int. Cl. A61K 9/00; A61K 9/16; A61K 31/192; A61P 9/04

(52) U.S. Cl. CPC A61K 9/006; A61K 9/1611; A61K 9/1617; A61K 9/1629; A61K 31/192; A61P 9/04

(57) ABSTRACT

Disclosed herein, in part, are pharmaceutical formulations for oral mucosal (e.g., sublingual and/or buccal) administration comprising micronized aspirin, (e.g., 91 mg to 200 mg micronized aspirin), a buffer, a surfactant, and one or more pharmaceutically acceptable excipients, wherein the pH of a solution of the formulation dissolved in 1 mL distilled water is less than or equal to 3. Methods of treating a subject in need thereof comprising administering one or more disclosed pharmaceutical formulations to the subject are also provided herein.

### CROSS-REFERENCE TO RELATED APPLICATIONS

This application claims the benefit of and priority to U.S. Provisional Application No. 63/702,381, filed Oct. 2, 2024, and U.S. Provisional Application No. 63/864,091, filed Aug. 14, 2025, each of which is incorporated by reference in its entirety herein.

### BACKGROUND

Aspirin is an established drug that has been in common use around the world for decades. Aspirin is a non-steroidal anti-inflammatory drug (NSAID) that is useful in treating and/or reducing many pathologies, including pain, fever, and inflammation. A subject suffering from a condition treatable with aspirin may benefit from the rapid delivery of aspirin, and there is thus a need to develop pharmaceutical formulations and/or dosage forms that allow for rapid and direct absorption of aspirin into a subject's systemic circulatory system where therapeutic effects for specific indications occur.

### SUMMARY

Described herein are compositions, pharmaceutical formulations, and dosage forms useful for delivering aspirin to a subject in need thereof, e.g., a subject suffering from a disease or disorder treatable with aspirin, as described herein. Also provided herein are methods of treating a subject suffering from a disease or disorder described herein, the methods comprising administering to the subject a formulation described herein. Also described herein are methods for making pharmaceutical formulations described herein.

In one aspect, provided herein is a pharmaceutical formulation suitable for oral mucosal (e.g., sublingual and/or buccal) administration comprising:
91 mg to 200 mg micronized aspirin;
a buffer;
a surfactant; and
one or more pharmaceutically acceptable excipients,
wherein the pH of a solution of the formulation dissolved in 1 ml distilled water is less than or equal to 3.

In another aspect, provided herein is a pharmaceutical formulation suitable for oral mucosal (e.g., sublingual and/or buccal) administration comprising:
91 mg to 200 mg micronized aspirin;
citric acid or a hydrate thereof;
tri-potassium citrate or a hydrate thereof;
sodium lauryl sulfate; and
one or more pharmaceutically acceptable excipients.

In another aspect, provided herein is a pharmaceutical formulation suitable for oral mucosal (e.g., sublingual and/or buccal) administration comprising:
91 mg to 200 mg micronized aspirin;
citric acid anhydrous;
tri-potassium citrate monohydrate;
sodium lauryl sulfate; and
one or more pharmaceutically acceptable excipients.

In another aspect, provided herein is a pharmaceutical formulation suitable for oral mucosal (e.g., sublingual and/or buccal) administration comprising:
162.5 mg micronized aspirin;
8.2 mg citric acid anhydrous;
2.4 mg tri-potassium citrate monohydrate
3.7 mg sodium lauryl sulfate; and
one or more pharmaceutically acceptable excipients.

In another aspect, provided herein is a pharmaceutical formulation suitable for oral mucosal (e.g., sublingual and/or buccal) administration comprising:
162.5 mg micronized aspirin;
0.82 mg citric acid anhydrous;
0.24 mg tri-potassium citrate monohydrate
3.65 mg sodium lauryl sulfate; and
one or more pharmaceutically acceptable excipients.

In another aspect, provided herein is a kit for suitable storage of an oral mucosal (e.g., sublingual and/or buccal) aspirin formulation comprising:
a pouch suitable for packaging a formulation disclosed herein; and
a formulation disclosed herein disposed within the pouch.

In another aspect, provided herein is a method of treating a disease, disorder, or condition in a subject in need thereof, the method comprising administering to the subject one or more formulations disclosed herein.

### BRIEF DESCRIPTION OF THE DRA WINGS

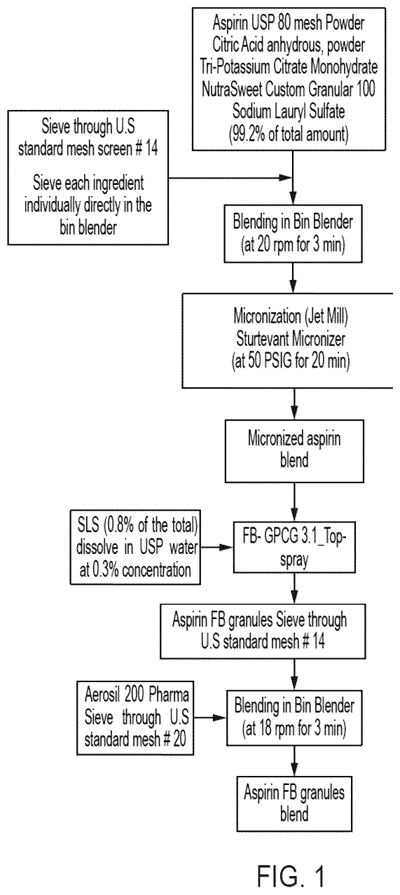

FIG. 1 is a flow chart describing an exemplary fluid bed granulation process for manufacturing aspirin formulations suitable for oral mucosal aspirin administration.

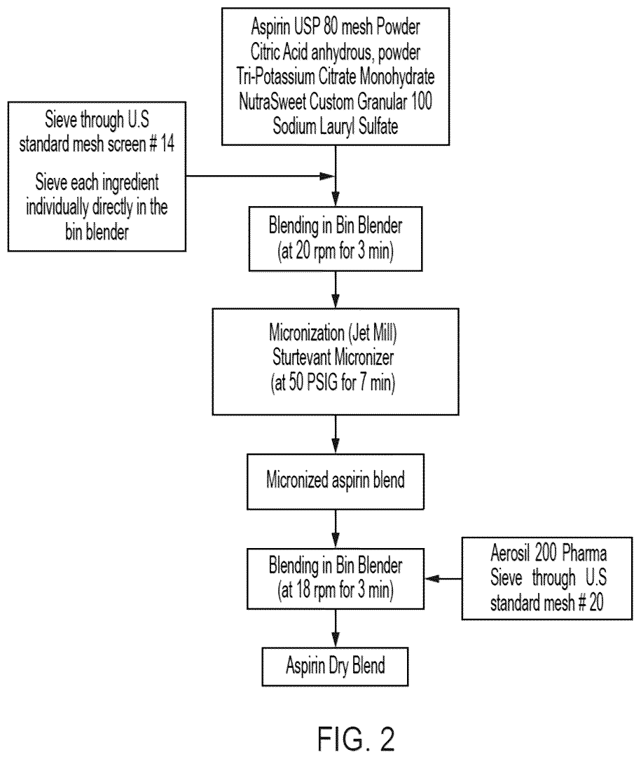

FIG. 2 is a flow chart describing an exemplary dry blend process for manufacturing aspirin formulations suitable for oral mucosal administration.

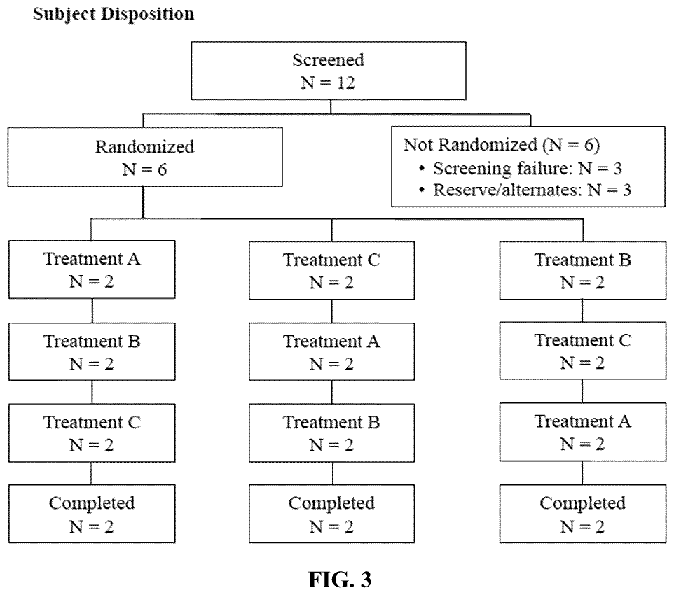

FIG. 3 shows the subject disposition data for the Phase 1 trial described in Example 5.

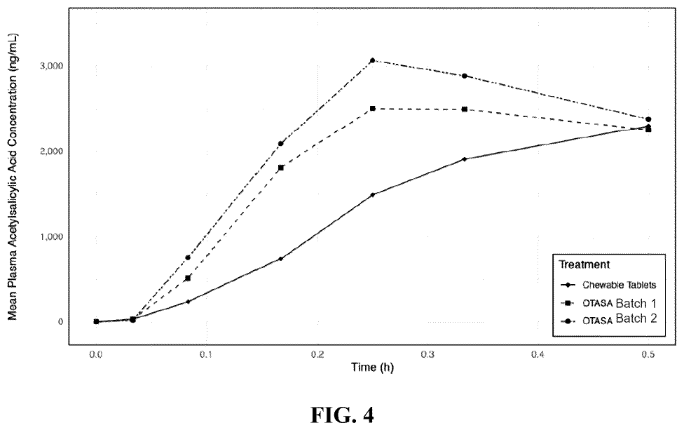

FIG. 4 is a graph showing the mean plasma concentration of ASA in the first 30 minutes post-dose from the Phase 1 trial described in Example 5.

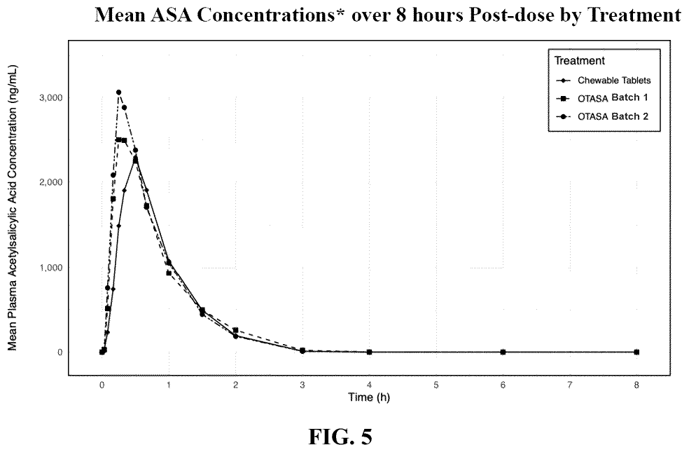

FIG. 5 is a graph showing the mean plasma concentration of ASA up to 8 hours post-dose from the Phase 1 trial described in Example 5. Mean plasma ASA concentrations 10 minutes (0.167 hours) following administration of OTASA Batch 1 and Batch 2 were significantly higher than those reported following administration of the chewable tablets (unadjusted p=0.023 and p=0.026, respectively, paired t-test, one-sided). The mean plasma concentration following administration of OTASA Batch 1 was also significantly higher than that of the chewed tablets at 15-minutes (0.25 hours) post-dose (p=0.029). Plasma ASA concentrations below the LLOQ (50.67 ng/mL) were imputed with zero.

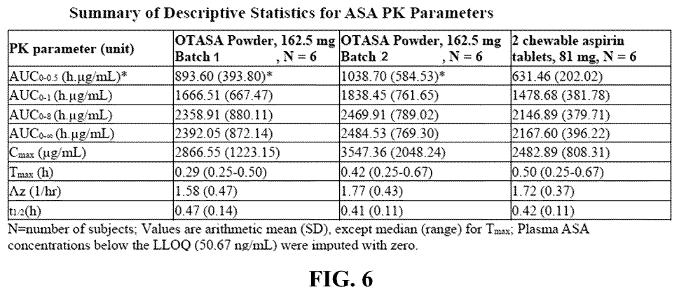

FIG. 6 is a table summarizing the individual ASA plasma PK parameters from the Phase 1 trial described in Example 5. Mean AUC₀₋₃₀ values following administration of OTASA Batch 1 and Batch 2 were significantly higher than following administration of the chewable uncoated tablets (unadjusted p=0.046 and p=0.049, respectively, paired t-test, one-sided).

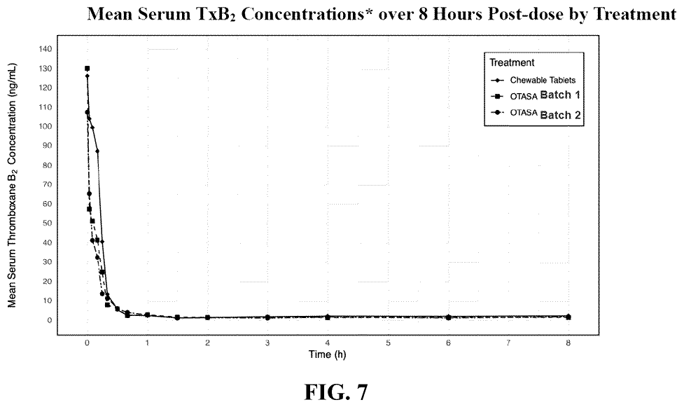

FIG. 7 is a graph showing the mean serum concentration of thromboxane B₂ up to 8 hours post-dose from the Phase 1 trial described in Example 5. Mean serum TxB2 concentrations 10 and 15 minutes (0.167 and 0.25 hours) following administration of OTASA Batch 2 were significantly lower than for the chewable tablets at the same timepoints (p=0.047 and p=0.032, respectively, paired t-test, one-sided). Serum TxB2 concentrations below the LLOQ (1.0 ng/ml) were imputed with 0.99 ng/mL.

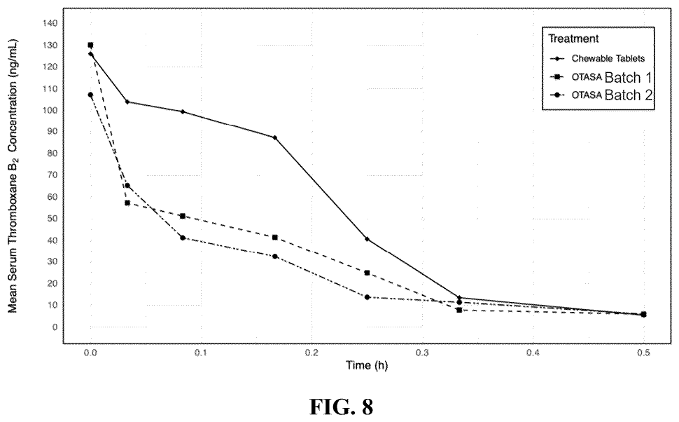

FIG. 8 is a graph showing the mean serum concentration of thromboxane B₂ in first 30 minutes post-dose from the Phase 1 trial described in Example 5.

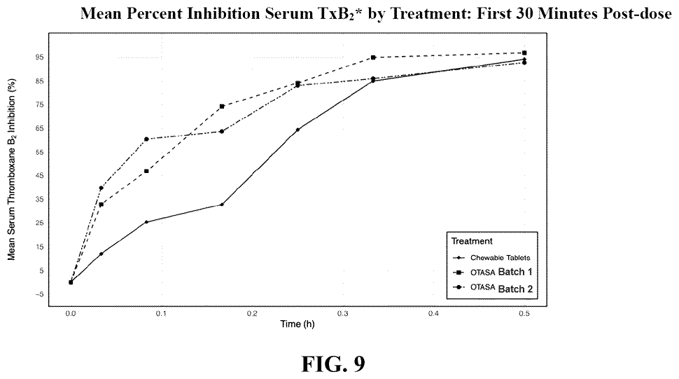

FIG. 9 is a graph showing the mean serum inhibition of thromboxane B₂ in first 30 minutes post-dose from the Phase 1 trial described in Example 5. Mean percent inhibition of serum TxB2 concentrations 2 minutes (0.033 hours) following administration of OTASA Batch 2 and 10 and 15 minutes (0.167 and 0.25 hours) following administration of OTASA Batch 1 were significantly higher than following administration of the chewable tablets (unadjusted p=0.012, p=0.038 and p=0.029, respectively, paired t-test, one-sided).

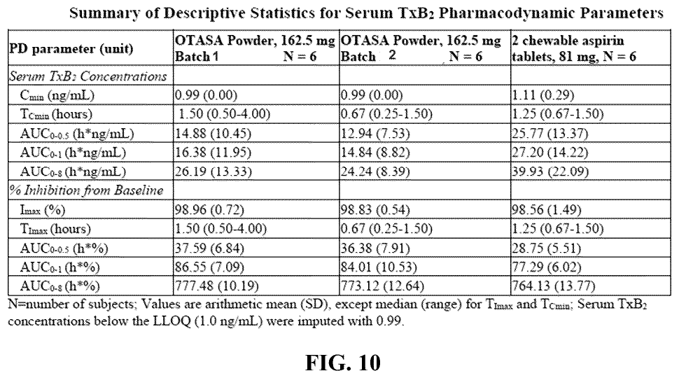

FIG. 10 is a table summarizing the individual serum TxB2 PD parameters with descriptive statistics from the Phase 1 trial described in Example 5.

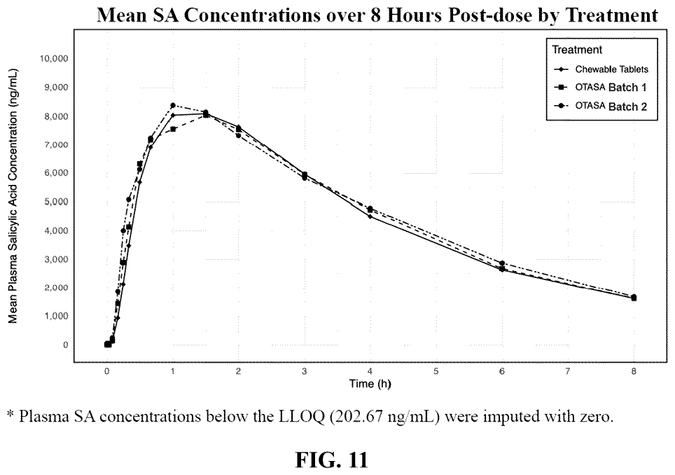

FIG. 11 is a graph showing the mean serum concentration of SA up to 8 hours after dosing from the Phase 1 trial described in Example 5.

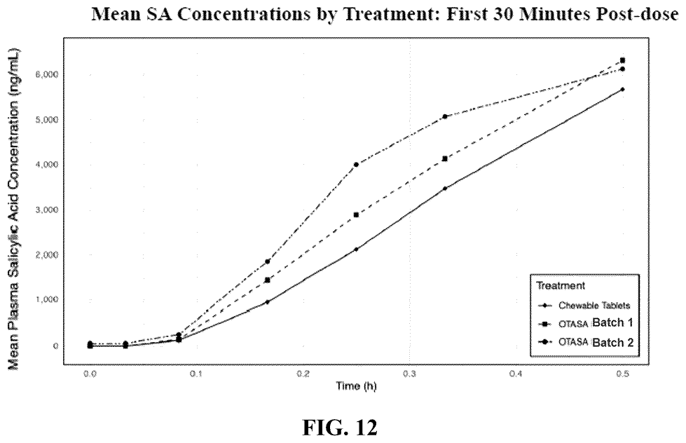

FIG. 12 is a graph showing the mean serum concentration of SA in the first 30 minutes post-dose from the Phase 1 trial described in Example 5.

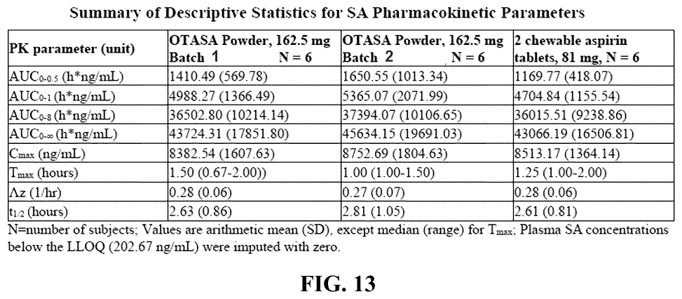

FIG. 13 is a table summarizing the individual serum SA plasma PK parameters with descriptive statistics from the Phase 1 trial described in Example 5.

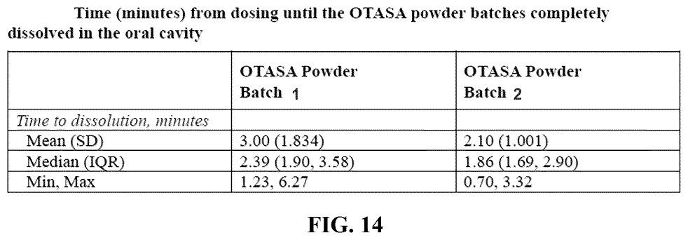

FIG. 14 is a table summarizing the individual times from dosing until each OTASA powder completely dissolved from the Phase 1 trial described in Example 5.

### DETAILED DESCRIPTION

As generally described herein, the present disclosure provides, in part, compositions, pharmaceutical formulations, and dosage forms useful for delivering aspirin to a subject in need thereof, e.g., a subject suffering from a disease, disorder, or condition treatable with aspirin, or a healthy subject prophylactically treatable with aspirin. The compositions, pharmaceutical formulations, and dosage forms described herein are formulated for oral mucosal (e.g., sublingual and/or buccal) delivery. Also provided herein are methods of treating a subject suffering from a disease, disorder, or condition described herein, the methods comprising administering to the subject one or more formulations described herein. The disclosure also provides methods for making the compositions, pharmaceutical formulations, and dosage forms described herein.

### Definitions

As used herein, “pharmaceutically acceptable excipient,” refers to a substance that aids the administration of an active agent to a subject by, for example, modifying the stability of an active agent or modifying the absorption by a subject upon administration. A pharmaceutically acceptable excipient typically has no significant adverse toxicological effect on the subject. Examples of pharmaceutically acceptable excipients include, for example, water, NaCl (including salt solutions), normal saline solutions, ½ normal saline, sucrose, glucose, bulking agents, buffers, binders, fillers, disintegrants, lubricants, coatings, sweeteners, flavors, alcohols, oils, gelatins, carbohydrates such as amylose or starch, fatty acid esters, hydroxymethycellulose, polyvinyl pyrrolidine, and colors, and the like. One of skill in the art will recognize that other pharmaceutical excipients known in the art are useful in the present invention and include those listed in, for example, the Handbook of Pharmaceutical Excipients, Rowe R. C., Shesky P. J., and Quinn M. E., 6th Ed., The Pharmaceutical Press, RPS Publishing (2009). The terms “bulking agent,” and “buffer” are used in accordance with the plain and ordinary meaning within the art.

As used herein, “surfactant” refers to a substance that reduces the surface tension of a liquid in which it is dissolved. In some embodiments, a surfactant comprises a water-soluble (hydrophilic) portion and a fat-soluble (lipophilic) portion. In some embodiments, surfactants may be anionic surfactants.

As used herein, “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺ (C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, a “hydrate” of a compound refers to an association or complex of one or more water molecules with the compound.

As used herein, a “subject” to which administration is contemplated includes, but is not limited to, humans (i.e., a male or female of any age group, e.g., a pediatric subject (e.g., adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

“Disease,” “disorder,” and “condition” are used interchangeably herein.

As used herein, and unless otherwise specified, the terms “treat,” “treating,” and “treatment” contemplate an action that occurs while a subject is suffering from the specified disease, disorder or condition or at risk for the specified disease, disorder, or condition, which reduces the severity of the disease, disorder or condition, or retards or slows the progression and/or emergence of the disease, disorder or condition.

As used herein, the “effective amount” of a compound refers to an amount sufficient to elicit the desired biological or physiologic response. As will be appreciated by those of ordinary skill in this art, the effective amount of a compound of the invention may vary depending on such factors as the desired biological endpoint, the pharmacokinetics of the compound, the disease being treated, the mode of administration, and the age, health, and condition of the subject.

An effective amount encompasses an amount sufficient for therapeutic and/or prophylactic treatment. A therapeutically effective amount and a prophylactically effective amount may differ in certain embodiments.

As used herein, the term “particle size” is defined as the diameter of a particle as determined by a particle size analyzer.

As used herein, the term “Cₘₐₓ” refers to the maximum plasma concentration of a compound described herein (e.g., aspirin) after administration of a formulation containing the compound (e.g., a formulation disclosed herein) to a subject. The term “Cₘₐₓ/D” refers to the Cₘₐₓ divided by the dose of the compound contained in the administered formulation.

As used herein, the term “Tₘₐₓ” refers to time to Cₘₐₓ following administration of a formulation described herein to a subject.

As used herein, the term “AUC” refers to the area under the plasma concentration vs. time curve from zero to infinity for a compound described herein (e.g., the area under the aspirin plasma concentration vs. time curve) after administration of a formulation containing the compound (e.g., a formulation disclosed herein) to a subject. The term “AUC/D” refers to the AUC divided by the dose of the compound contained in the administered formulation.

### Compounds

Described herein are pharmaceutical formulations or dosage forms comprising aspirin, represented by formula (I):

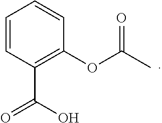

The compound of formula (I) may be referred to as aspirin, 2-acetoxybenzoic acid, acetylsalicylate, acetylsalicylic acid, and ASA, and these terms are used interchangeably herein. Aspirin may exist in a crystalline solid form comprising white crystals, commonly tabular or needle-like, or white crystalline powder. Aspirin has a melting point of 135° C. (PubChem).

Aspirin contains a carboxylic acid group and has a pKa of approximately 3.5 (PubChem). Aspirin has a log p value of 1.19 (PubChem).

### Pharmaceutical Formulations and Dosage Forms

In one aspect, the present disclosure provides pharmaceutical formulations or dosage forms useful for preventing and/or treating a disease, disorder, or condition described herein. The disclosure provides pharmaceutical formulations suitable for oral mucosal (e.g., sublingual and/or buccal) delivery that contain aspirin as the active ingredient, and pharmaceutically acceptable excipients that include a buffer and a surfactant.

Oral mucosal (e.g., sublingual and/or buccal) delivery utilizes the many capillaries found in the cheek and under the tongue (e.g., within the oral cavity) to quickly introduce a drug into the circulatory system of a subject. Oral mucosal (e.g., sublingual and/or buccal) delivery has the added benefit of precluding direct physical/chemical contact with digestive tract mucosa and avoiding first pass metabolism by the digestive tract and the liver. Oral mucosal delivery is a preferred delivery method for drugs that do not absorb efficiently in the stomach and/or digestive tract, including the rectum, or that cannot be given intravenously, subcutaneously, intramuscularly and/or intrathecally.

Oral mucosal (e.g., sublingual and/or buccal) delivery of aspirin allows for rapid introduction of the drug into the circulatory system of a subject in need thereof. This approach represents a significant improvement in subject outcomes due to aspirin's antithrombotic, antipyretic, and anti-inflammatory properties, and associated use in treating conditions in which a subject would benefit from rapid and effective delivery of aspirin. An exemplary subject that would benefit from rapid delivery of aspirin is a geriatric orthopedic surgery subject who fails a swallow test and prefers not to be injected with low molecular weight heparin and is in need of venous thrombosis prophylaxis.

Aspirin contains a carboxylic acid functional group and has a pKa of approximately 3.5. In aqueous solution, aspirin is in equilibrium with its conjugate base. This acid-conjugate base equilibrium is represented by Equation 1 below.

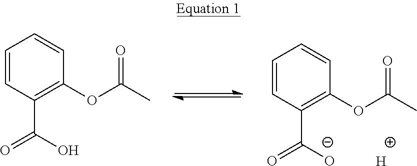

In its conjugate base/ionized form, aspirin is unable to efficiently cross the mucous membranes in the oral cavity to enter the blood stream, rendering oral mucosal (e.g., sublingual and/or buccal) delivery of ionized aspirin inefficient. The pH of saliva in the oral cavity ranges between 6.2 to 7.6 (Dawood, I. M.; El-Samarrai, S. K. Saliva and Oral Health. Int. J. Adv. Res. Biol. Sci. 2018, 5, 1-45) depending on the individual, with an average pH of 6.7. The Henderson-Hasselbalch equation, represented by Equation 2 below, provides the ratio of conjugate base to acid at a given pH of an acid having a given pKa.

$$pH = {pK}_{a} + {log}_{10} ( \frac{[ Base ]}{[ Acid ]} ) \qquad Equation 2$$

Thus, in an aqueous environment having a pH of 6.7 (i.e., commensurate to the pH of the saliva of an average human subject), aspirin, having a pKa of 3.5, would exist in its ionized/conjugate base form at a ratio of 1584 molecules of ionized conjugate base relative to 1 molecule of non-ionized acid and would be unable to efficiently enter a subject's circulatory system through oral mucosal (e.g., sublingual and/or buccal) routes. Exemplary calculations are provided below.

Using the lower saliva pH limit of 6.2 in equation 1:

$$6.2 = 3.5 + {log}_{10} ( [ {A}^{-} ] / [ HA ] )$$

$$Thus , {log}_{10} ( [ {A}^{-} ] / [ HA ] ) = 2.7$$

$$Therefore , [ {A}^{-} ] / [ HA ] = 1 {0}^{2.7} = 501$$

For every one molecule of non-ionized aspirin that can enter the blood stream, there are 501 ionized aspirin molecules that cannot cross the mucosal membranes to enter the sublingual capillaries.

Using the upper saliva pH limit of 7.6 in equation 1:

$$7.6 = 3.5 + {log}_{10} ( [ {A}^{-} ] / [ HA ] )$$

$$Thus , {log}_{10} ( [ {A}^{-} ] / [ HA ] ) = 4.1$$

$$Therefore , [ {A}^{-} ] / [ HA ] = 1 {0}^{4.1} = 12589$$

For every one molecule of non-ionized aspirin that can enter the blood stream, there are 12589 ionized aspirin molecules that cannot cross the mucosal membranes to enter the sublingual capillaries.

Using the average saliva pH value of 6.7 in equation 1:

$$6.7 = 3.5 + {log}_{10} ( [ {A}^{-} ] / [ HA ] )$$

$$Thus , {log}_{10} ( [ {A}^{-} ] / [ HA ] ) = 3.2$$

$$Therefore , [ {A}^{-} ] / [ HA ] = 1 {0}^{3.2} = 1584$$

For every one molecule of non-ionized aspirin that can enter the blood stream, there are 1584 ionized aspirin molecules that cannot cross the mucosal membranes to enter the sublingual capillaries.

Disclosed herein are pharmaceutical formulations suitable for the oral mucosal (e.g., sublingual and/or buccal) delivery of aspirin that overcome problems associated with the relatively high pH of the oral cavity and resulting ionization of aspirin. The pharmaceutical formulations of the present disclosure comprise aspirin, a buffer, a surfactant, and one or more additional pharmaceutically acceptable excipients. When administered to a subject through the oral mucosa (e.g., sublingually and/or buccally), the buffer component reduces the local pH within the oral cavity and maintains a reduced pH throughout the duration of drug delivery. The reduced pH causes the aspirin to exist primarily in its non-ionized form throughout administration, thus enabling delivery across the mucous membranes of the oral cavity and directly into the circulatory system of the subject. In some embodiments, the buffer component may also stimulate salivation, thus increasing dissolution and absorption of the aspirin.

The surfactant (e.g., sodium lauryl sulfate), spontaneously forms micellar structures in aqueous media. Lipophilic tails of the surfactant molecules orient inside the micelle, and the micelle is stabilized by hydrophobic interactions among the lipophilic tails. Polar groups of the surfactant molecules orient outward toward the surrounding aqueous environment where they stabilize the micelle through hydrogen bonding with water and other ionic interactions with cations such as sodium, potassium, and calcium. During the spontaneous formation of micelles, aspirin molecules become trapped and encapsulated within the micelles. Aspirin within the micelles is in its neutral form due to low pH generated by the buffer component. The micelles promote direct systemic absorption of aspirin. The surfactant also aids in formulation dissolution and promotes absorption of the aspirin by reducing salivary protein binding of the aspirin. Concurrently, the repulsion of anionic aspirin molecules by the surfactant and surfactant micelles pushes the aspirin acid-conjugate base equilibrium toward the acidic undissociated form of aspirin (i.e., its neutral form), thereby favoring absorption through mucous membranes in the oral cavity. The formulations disclosed herein comprise surfactant in amounts that meet or exceed the critical micelle concentration (CMC) for surfactant micelle formation in saliva.

Manufacturing/processing techniques (e.g., micronizing the aspirin and other ingredients in the formulation) increase the total surface area of the formulation components, thereby increasing drug dissolution and absorption, and corresponding bioavailability. Exemplary manufacturing/processing techniques useful for preparing the formulations are disclosed herein.

In one aspect, provided herein is a pharmaceutical formulation suitable for oral mucosal (e.g., sublingual and/or buccal) administration comprising:
91 mg to 200 mg micronized aspirin;
a buffer;
a surfactant;
and one or more pharmaceutically acceptable excipients,
wherein the pH of a solution of the formulation dissolved in 1 mL distilled water is less than or equal to 3.

In another aspect, provided herein is a pharmaceutical formulation suitable for oral mucosal (e.g., sublingual and/or buccal) administration comprising:
91 mg to 200 mg aspirin;
a buffer;
a surfactant;
and one or more pharmaceutically acceptable excipients,
wherein the pH of a solution of the formulation dissolved in 1 mL distilled water is less than or equal to 3.

In some embodiments, the formulation comprises granules comprising an intragranular fraction, wherein the intragranular fraction comprises the aspirin. In some embodiments, the intragranular fraction further comprises the buffer. In some embodiments, the intragranular fraction further comprises one or more pharmaceutically acceptable excipients (e.g., sodium lauryl sulfate and/or aspartame).

In some embodiments, the formulation does not comprise granules.

In some embodiments, the pH of a solution of the formulation dissolved in 1 ml distilled water is 2 to 3, 2 to 2.9, 2 to 2.8, 2 to 2.7, 2 to 2.6, 2 to 2.5, 2 to 2.4, 2 to 2.3, or 2 to 2.2. In some embodiments, the pH of a solution of the formulation dissolved in 1 mL distilled water is 2 to 3, 2.1 to 3, 2.2 to 3, 2.3 to 3, 2.4 to 3, 2.5 to 3, 2.6 to 3, 2.7 to 3, 2.8 to 3, or 2.9 to 3.

In some embodiments, the pH of a solution of the formulation dissolved in 1 mL saliva is 2 to 3, 2 to 2.9, 2 to 2.8, 2 to 2.7, 2 to 2.6, 2 to 2.5, 2 to 2.4, 2 to 2.3, or 2 to 2.2. In some embodiments, the pH of a solution of the formulation dissolved in 1 mL saliva is 2 to 3, 2.1 to 3, 2.2 to 3, 2.3 to 3, 2.4 to 3, 2.5 to 3, 2.6 to 3, 2.7 to 3, 2.8 to 3, or 2.9 to 3.

In some embodiments, the pH of a solution of the formulation dissolved in 1 mL distilled water is 2, 2.1, 2.2, 2.3, 2.4, 2.5, 2.6, 2.7, 2.8, 2.9, or 3.

In some embodiments, the pH of a solution of the formulation dissolved in 1 mL saliva is 2, 2.1, 2.2, 2.3, 2.4, 2.5, 2.6, 2.7, 2.8, 2.9, or 3.

In some embodiments, the pH of a solution of the formulation dissolved in 1 ml distilled water is less than 2.9. In some embodiments, the pH of a solution of the formulation dissolved in 1 ml distilled water is less than 2.8. In some embodiments, the pH of a solution of the formulation dissolved in 1 ml distilled water is less than 2.7. In some embodiments, the pH of a solution of the formulation dissolved in 1 mL distilled water is less than 2.6. In some embodiments, the pH of a solution of the formulation dissolved in 1 mL distilled water is less than 2.5. In some embodiments, the pH of a solution of the formulation dissolved in 1 ml distilled water is less than 2.4. In some embodiments, the pH of a solution of the formulation dissolved in 1 ml distilled water is less than 2.3. In some embodiments, the pH of a solution of the formulation dissolved in 1 ml distilled water is less than 2.2. In some embodiments, the pH of a solution of the formulation dissolved in 1 mL distilled water is less than 2.1. In some embodiments, the pH of a solution of the formulation dissolved in 1 mL distilled water is 2 to 2.5. In some embodiments, the pH of a solution of the formulation dissolved in 1 ml distilled water is 1.8 to 2.5.

In some embodiments, the pH of a solution of the formulation dissolved in 1 mL saliva is less than 2.9. In some embodiments, the pH of a solution of the formulation dissolved in 1 mL saliva is less than 2.8. In some embodiments, the pH of a solution of the formulation dissolved in 1 mL saliva is less than 2.7. In some embodiments, the pH of a solution of the formulation dissolved in 1 mL saliva is less than 2.6. In some embodiments, the pH of a solution of the formulation dissolved in 1 mL saliva is less than 2.5. In some embodiments, the pH of a solution of the formulation dissolved in 1 mL saliva is less than 2.4. In some embodiments, the pH of a solution of the formulation dissolved in 1 mL saliva is less than 2.3. In some embodiments, the pH of a solution of the formulation dissolved in 1 mL saliva is less than 2.2. In some embodiments, the pH of a solution of the formulation dissolved in 1 mL saliva is less than 2.1. In some embodiments, the pH of a solution of the formulation dissolved in 1 mL saliva is 2 to 2.5. In some embodiments, the pH of a solution of the formulation dissolved in 1 mL saliva is 1.8 to 2.5.

In some embodiments, the pH of a solution of the formulation dissolved in 1 ml distilled water is equal to or greater than 2.7 and less than or equal to 3. In some embodiments, the pH of a solution of the formulation dissolved in 1 mL distilled water is equal to or greater than 2.8 and less than or equal to 3. In some embodiments, the pH of a solution of the formulation dissolved in 1 mL distilled water is equal to or greater than 2.9 and less than or equal to 3.

In some embodiments, the pH of a solution of the formulation dissolved in 1 mL saliva is equal to or greater than 2.7 and less than or equal to 3. In some embodiments, the pH of a solution of the formulation dissolved in 1 mL saliva is equal to or greater than 2.8 and less than or equal to 3. In some embodiments, the pH of a solution of the formulation dissolved in 1 mL saliva is equal to or greater than 2.9 and less than or equal to 3.

Determination of the pH of a solution of a formulation disclosed herein may be accomplished by methods known in the art, e.g., as described herein and by standard protocols, e.g., by protocols described in the United States Pharmacopocia, Chapter <791>.

In some embodiments, the buffer concentration of the formulation is at least 25 mM, at least 30 mM, at least 40 mM, at least 50 mM, at least 75 mM, at least 100 mM, at least 125 mM, at least 150 mM, at least 200 mM, at least 225 mM, at least 250 mM, at least 300 mM, at least 325 mM, at least 350 mM, at least 375 mM, at least 400 mM, at least 425 mM, at least 450 mM, at least 475 mM, or at least 500 mM when dissolved in 1 mL of distilled and/or deionized water.

In some embodiments, the buffer concentration of the formulation is 25 mM to 500 mM, 30 mM to 500 mM, 40 mM to 500 mM, 50 mM to 500 mM, 100 mM to 500 mM, 150 mM to 500 mM, 200 mM to 500 mM, 250 mM to 500 mM, 300 mM to 500 mM, 350 mM to 500 mM, 400 mM to 500 mM, or 450 mM to 500 mM when dissolved in 1 mL of distilled and/or deionized water. In some embodiments, the buffer concentration of the formulation is 50 mM to 500 mM, 50 mM to 450 mM, 50 mM to 400 mM, 50 mM to 350 mM, 50 mM to 300 mM, 50 mM to 250 mM, 50 mM to 200 mM, 50 mM to 150 mM, 50 mM to 100 mM, 25 mM to 100 mM, 25 mM to 75 mM, or 25 mM to 50 mM when dissolved in 1 mL of distilled and/or deionized water. In some embodiments, the buffer concentration of the formulation is 50 mM to 500 mM, 100 mM to 450 mM, 150 mM to 400 mM, or 200 mM to 350 mM when dissolved in 1 mL of distilled and/or deionized water.

In some embodiments, the buffer concentration of the formulation is 25 mM, 30 mM, 40 mM, 50 mM, 100 mM, 125 mM, 150 mM, 175 mM, 200 mM, 225 mM, 250 mM, 275 mM, 300 mM, 325 mM, 350 mM, 375 mM, 400 mM, 425 mM, 450 mM, 475 mM, or 500 mM when dissolved in 1 mL of distilled and/or deionized water.

In some embodiments, the buffer concentration of the formulation is at least 25 mM when dissolved in 1 mL of distilled and/or deionized water. In some embodiments, the buffer concentration of the formulation is at least 30 mM when dissolved in 1 mL of distilled and/or deionized water. In some embodiments, the buffer concentration of the formulation is at least 40 mM when dissolved in 1 mL of distilled and/or deionized water. In some embodiments, the buffer concentration of the formulation is at least 50 mM when dissolved in 1 mL of distilled and/or deionized water. In some embodiments, the buffer concentration of the formulation is at least 100 mM when dissolved in 1 mL of distilled and/or deionized water. In some embodiments, the buffer concentration of the formulation is at least 150 mM when dissolved in 1 mL of distilled and/or deionized water. In some embodiments, the buffer concentration of the formulation is at least 200 mM when dissolved in 1 mL of distilled and/or deionized water. In some embodiments, the buffer concentration of the formulation is at least 250 mM when dissolved in 1 mL of distilled and/or deionized water. In some embodiments, the buffer concentration of the formulation is at least 300 mM when dissolved in 1 mL of distilled and/or deionized water. In some embodiments, the buffer concentration of the formulation is at least 350 mM when dissolved in 1 mL of distilled and/or deionized water. In some embodiments, the buffer concentration of the formulation is at least 400 mM when dissolved in 1 mL of distilled and/or deionized water. In some embodiments, the buffer concentration of the formulation is at least 450 mM when dissolved in 1 mL of distilled and/or deionized water. In some embodiments, the buffer concentration of the formulation is at least 500 mM when dissolved in 1 mL of distilled and/or deionized water.

In some embodiments, the buffer concentration of the formulation is 25 mM when dissolved in 1 mL of distilled and/or deionized water. In some embodiments, the buffer concentration of the formulation is 30 mM when dissolved in 1 mL of distilled and/or deionized water. In some embodiments, the buffer concentration of the formulation is 40 mM when dissolved in 1 mL of distilled and/or deionized water. In some embodiments, the buffer concentration of the formulation is 50 mM when dissolved in 1 mL of distilled and/or deionized water. In some embodiments, the buffer concentration of the formulation is 100 mM when dissolved in 1 mL of distilled and/or deionized water. In some embodiments, the buffer concentration of the formulation is 150 mM when dissolved in 1 mL of distilled and/or deionized water. In some embodiments, the buffer concentration of the formulation is 200 mM when dissolved in 1 mL of distilled and/or deionized water. In some embodiments, the buffer concentration of the formulation is 250 mM when dissolved in 1 mL of distilled and/or deionized water. In some embodiments, the buffer concentration of the formulation is 300 mM when dissolved in 1 mL of distilled and/or deionized water. In some embodiments, the buffer concentration of the formulation is 350 mM when dissolved in 1 mL of distilled and/or deionized water. In some embodiments, the buffer concentration of the formulation is 400 mM when dissolved in 1 mL of distilled and/or deionized water. In some embodiments, the buffer concentration of the formulation is 450 mM when dissolved in 1 mL of distilled and/or deionized water. In some embodiments, the buffer concentration of the formulation is 500 mM when dissolved in 1 mL of distilled and/or deionized water.

As used herein, dissolution of a formulation of the disclosure encompasses both full and partial dissolution of the formulation in a solvent (e.g., distilled water). Solutions of the formulations disclosed herein encompass both mixtures in which the formulation has fully dissolved and mixtures in which the formulation has partially dissolved. In some embodiments, the formulation is at least 50%, at least 55%, at least 60%, at least 65%, at least 70%, at least 75%, at least 80%, at least 85%, at least 90%, or at least 95% dissolved in the solutions described herein. In some embodiments, the formulation is at least 90% dissolved in the solutions described herein. In some embodiments, the formulation is at least 95% dissolved in the solutions described herein. Percent dissolution is the fraction of the dose that has gone into solution within a specified time period as measured by a dissolution test as described herein.

In some embodiments, the formulation comprises 91 mg to 100 mg aspirin, 100 mg to 110 mg aspirin, 110 mg to 120 mg aspirin, 120 mg to 130 mg aspirin, 130 mg to 140 mg aspirin, 140 mg to 150 mg aspirin, 150 mg to 160 mg aspirin, 160 mg to 170 mg aspirin, 170 mg to 180 mg aspirin, 180 mg to 190 mg aspirin, 190 mg to 200 mg aspirin, 100 mg to 120 mg aspirin, 120 mg to 140 mg aspirin, 140 mg to 160 mg aspirin, 160 mg to 180 mg aspirin, 180 mg to 200 mg aspirin, 150 mg to 170 mg aspirin, 91 mg to 150 mg aspirin, or 150 mg to 200 mg aspirin. In some embodiments, the formulation comprises 100 mg aspirin, 110 mg aspirin, 120 mg aspirin, 130 mg aspirin, 140 mg aspirin, 150 mg aspirin, 160 mg aspirin, 170 mg aspirin, 180 mg aspirin, 190 mg aspirin, or 200 mg aspirin. In some embodiments, the formulation comprises 100 mg to 200 mg aspirin. In some embodiments, the formulation comprises 150 mg to 200 mg aspirin.

In some embodiments, the formulation comprises 91 mg aspirin, 95 mg aspirin, 100 mg aspirin, 105 mg aspirin, 110 mg aspirin, 115 mg aspirin, 120 mg aspirin, 125 mg aspirin, 130 mg aspirin, 135 mg aspirin, 140 mg aspirin, 145 mg aspirin, 150 mg aspirin, 155 mg aspirin, 160 mg aspirin, 165 mg aspirin, 170 mg aspirin, 175 mg aspirin, 180 mg aspirin, 185 mg aspirin, 190 mg aspirin, 195 mg aspirin, or 200 mg aspirin.

In some embodiments, the formulation comprises 100 mg to 163 mg aspirin. In some embodiments, the formulation comprises 145 mg to 163 mg aspirin. In certain embodiments, the formulation comprises 163 mg aspirin. In certain embodiments, the formulation comprises 162.5 mg aspirin.

In some embodiments, the micronized aspirin in the formulation has a median particle size of 1 μm to 20 μm, 1 μm to 18 μm, 1 μm to 16 μm, 1 μm to 14 μm, 1 μm to 12 μm, 1 μm to 10 μm, 1 μm to 8 μm, 1 μm to 6 μm, or 1 μm to 4 μm. In some embodiments, the micronized aspirin in the formulation has a median particle size of 1 μm to 10 μm. In some embodiments, the micronized aspirin in the formulation has a median particle size of less than 10 μm.

Determination of particle size may be accomplished by methods known in the art, e.g., by using a particle size analyzer and suspending the particles in a liquid phase in which they are insoluble or sparingly soluble, such as, e.g., an alkane such as, e.g., hexane.

In some embodiments, the buffer comprises citric acid, or a hydrate thereof, and a citrate salt, or a hydrate thereof. In some embodiments, the citric acid or hydrate thereof is selected from citric acid monohydrate and citric acid anhydrous. In certain embodiments, the citric acid or hydrate thereof is citric acid anhydrous. In certain embodiments, the citric acid or hydrate thereof is citric acid monohydrate. In some embodiments, the citrate salt or hydrate thereof is selected from the group consisting of monosodium citrate, disodium citrate, trisodium citrate, monopotassium citrate, dipotassium citrate, and tri-potassium citrate, or a hydrate thereof. In certain embodiments, the citrate salt or hydrate thereof is tri-potassium citrate monohydrate. In certain embodiments, the citrate salt or hydrate thereof is mono-potassium citrate or a hydrate thereof.

In some embodiments, the buffer consists of citric acid, or a hydrate thereof, and a citrate salt, or a hydrate thereof. In some embodiments, the citric acid or hydrate thereof is selected from citric acid monohydrate and citric acid anhydrous. In certain embodiments, the citric acid or hydrate thereof is citric acid anhydrous. In certain embodiments, the citric acid or hydrate thereof is citric acid monohydrate. In some embodiments, the citrate salt or hydrate thereof is selected from the group consisting of monosodium citrate, disodium citrate, trisodium citrate, monopotassium citrate, dipotassium citrate, and tri-potassium citrate, or a hydrate thereof. In certain embodiments, the citrate salt or hydrate thereof is tri-potassium citrate monohydrate. In certain embodiments, the citrate salt or hydrate thereof is mono-potassium citrate or a hydrate thereof.

In some embodiments, the buffer is 4% to 25%, 8% to 25%, 12% to 25%, 16% to 25%, 4% to 20%, 4% to 16%, 4% to 12%, or 4% to 8% of the formulation by mass. In certain embodiments, the buffer is 4% to 8% of the formulation by mass. In some embodiments, the buffer is 4%, 6%, 8%, 10%, 12%, 14%, 16%, 18%, 20%, 22%, 24%, or 25% of the formulation by mass. In certain embodiments, the buffer is 6% of the formulation by mass. In some embodiments, the buffer is 5.5% of the formulation by mass.

In some embodiments, the surfactant is an anionic surfactant. In some embodiments, the surfactant is selected from the group consisting of sodium lauryl sulfate, ammonium lauryl sulfate, potassium lauryl sulfonate, potassium lauryl phosphate, sodium deoxycholate, and potassium deoxycholate. In certain embodiments, the surfactant is sodium lauryl sulfate.

In some embodiments, the anionic surfactant (e.g., sodium lauryl sulfate) increases the percentage of oral mucosal (e.g., sublingual and/or buccal) absorption of aspirin in a subject administered the formulation oral mucosally (e.g., sublingually and/or buccally) compared to a subject administered an oral mucosal aspirin formulation that does not comprise an anionic surfactant.

In some embodiments, the anionic surfactant increases the percentage of oral mucosal (e.g., sublingual and/or buccal) absorption of aspirin in a subject administered the formulation oral mucosally (e.g., sublingually and/or buccally) compared to a subject administered an oral mucosal aspirin formulation that does not comprise an anionic surfactant by 1% to 5%, 5% to 10%, 10% to 15%, or 15% to 20%. In some embodiments, the anionic surfactant increases by 10% to 20% the percentage of oral mucosal (e.g., sublingual and/or buccal) absorption of aspirin in a subject administered the formulation oral mucosally (e.g., sublingually and/or buccally) compared to a subject administered an oral mucosal aspirin formulation that does not comprise an anionic surfactant. In some embodiments, the anionic surfactant increases by at least 10% or at least 20% the percentage of oral mucosal (e.g., sublingual and/or buccal) absorption of aspirin in a subject administered the formulation oral mucosally (e.g., sublingually and/or buccally) compared to a subject administered an oral mucosal aspirin formulation that does not comprise an anionic surfactant.

In some embodiments, the surfactant is 1.6% to 5%, 1.8% to 5%, 2% to 5%, 2.2% to 5%, 2.4% to 5%, 2.6% to 5%, 2.8% to 5%, 3% to 5%, 1.6% to 4.8%, 1.6% to 4.6%, 1.6% to 4.4%, 1.6% to 4.2%, 1.6% to 4%, 1.6% to 3.8%, 1.6% to 3.6%, 1.8% to 4.8%, 2% to 4.6%, 2.2% to 4.4%, 2.4% to 4.2%, 2.6% to 4%, or 2.8% to 3.8% of the formulation by mass. In certain embodiments, the surfactant is 1.6% to 3% of the formulation by mass. In certain embodiments, the formulation is 1.9% of the formulation by mass. In some embodiments, the surfactant is 1.6%, 1.8%, 2.0%, 2.2%, 2.4%, 2.6%, 2.8%, 3%, 3.2%, 3.4%, 3.6%, 3.8%, 4%, 4.2%, 4.4%, 4.6%, 4.8%, or 5% of the formulation by mass.

In another aspect, provided herein is a pharmaceutical formulation suitable for oral mucosal (e.g., sublingual and/or buccal) administration comprising:
91 mg to 200 mg micronized aspirin;
citric acid or a hydrate thereof;
tri-potassium citrate or a hydrate thereof;
sodium lauryl sulfate; and
one or more pharmaceutically acceptable excipients.

In another aspect, provided herein is a pharmaceutical formulation suitable for oral mucosal (e.g., sublingual and/or buccal) administration comprising:
91 mg to 200 mg aspirin;
citric acid or a hydrate thereof;
tri-potassium citrate or a hydrate thereof;
sodium lauryl sulfate; and
one or more pharmaceutically acceptable excipients.

In some embodiments, the formulation comprises 8 mg to 75 mg citric acid, 10 mg to 75 mg citric acid, 15 mg to 75 mg citric acid, 20 mg to 75 mg citric acid, 25 mg to 75 mg citric acid, 30 mg to 75 mg citric acid, 35 mg to 75 mg citric acid, 40 mg to 75 mg citric acid, 45 mg to 75 mg citric acid, 50 mg to 75 mg citric acid, 55 mg to 75 mg citric acid, 60 mg to 75 mg citric acid, 8 mg to 70 mg citric acid, 8 mg to 65 mg citric acid, 8 mg to 60 mg citric acid, 8 mg to 55 mg citric acid, 8 mg to 50 mg citric acid, 8 mg to 45 mg citric acid, 8 mg to 40 mg citric acid, 8 mg to 35 mg citric acid, 8 mg to 30 mg citric acid, 8 mg to 25 mg citric acid, 8 mg to 20 mg citric acid, 8 mg to 15 mg citric acid, 10 mg to 70 mg citric acid, 15 mg to 65 mg citric acid, 20 mg to 60 mg citric acid, 25 mg to 55 mg citric acid, 30 mg to 50 mg citric acid, or 35 mg to 45 mg citric acid, or an equivalent molar amount of a hydrate thereof. In some embodiments, the formulation comprises 8 mg to 75 mg citric acid, or an equivalent molar amount of a hydrate thereof. In some embodiments, the formulation comprises 8 mg to 15 mg citric acid, or an equivalent molar amount of a hydrate thereof.

In some embodiments, the formulation comprises 8 mg citric acid, 10 mg citric acid, 15 mg citric acid, 20 mg citric acid, 25 mg citric acid, 30 mg citric acid, 35 mg citric acid, 40 mg citric acid, 45 mg citric acid, 50 mg citric acid, 55 mg citric acid, 60 mg citric acid, 65 mg citric acid, 70 mg citric acid, or 75 mg citric acid, or an equivalent molar amount of a hydrate thereof. In certain embodiments, the formulation comprises 8.2 mg citric acid, or an equivalent molar amount of a hydrate thereof.

In some embodiments, the formulation comprises 0.1 mg citric acid, 0.2 mg citric acid, 0.3 mg citric acid, 0.4 mg citric acid, 0.5 mg citric acid, 0.6 mg citric acid, 0.7 mg citric acid, 0.8 mg citric acid, 0.9 mg citric acid, or 1.0 mg citric acid, or an equivalent molar amount of a hydrate thereof. In certain embodiments, the formulation comprises 0.82 mg citric acid, or an equivalent molar amount of a hydrate thereof.

In some embodiments, the formulation comprises 2.1 mg to 20 mg tri-potassium citrate, 4 mg to 20 mg tri-potassium citrate, 6 mg to 20 mg tri-potassium citrate, 8 mg to 20 mg tri-potassium citrate, 10 mg to 20 mg tri-potassium citrate, 12 mg to 20 mg tri-potassium citrate, 14 mg to 20 mg tri-potassium citrate, 16 mg to 20 mg tri-potassium citrate, 18 mg to 20 mg tri-potassium citrate, 2.1 mg to 19 mg, tri-potassium citrate, 2.1 mg to 17 mg tri-potassium citrate, 2.1 mg to 15 mg tri-potassium citrate, 2.1 mg to 13 mg tri-potassium citrate, 2.1 mg to 11 mg tri-potassium citrate, 2.1 mg to 9 mg tri-potassium citrate, 2.1 mg to 7 mg tri-potassium citrate, 2.1 mg to 5 mg tri-potassium citrate, 2 mg to 20 mg tri-potassium citrate, 4 mg to 19 mg tri-potassium citrate, 6 mg to 17 mg tri-potassium citrate, 8 mg to 15 mg tri-potassium citrate, or 10 mg to 13 mg tri-potassium citrate, or an equivalent molar amount of a hydrate thereof. In some embodiments, the formulation comprises 2.1 mg to 20 mg tri-potassium citrate, or an equivalent molar amount of a hydrate thereof. In some embodiments, the formulation comprises 0.3 mg to 2.7 mg tri-potassium citrate, or an equivalent molar amount of a hydrate thereof.

In some embodiments, the formulation comprises 2.1 mg tri-potassium citrate, 3 mg tri-potassium citrate, 5 mg tri-potassium citrate, 7 mg tri-potassium citrate, 9 mg tri-potassium citrate, 11 mg tri-potassium citrate, 13 mg tri-potassium citrate, 15 mg tri-potassium citrate, 17 mg tri-potassium citrate, 19 mg tri-potassium citrate, or 20 mg tri-potassium citrate, or an equivalent molar amount of a hydrate thereof. In certain embodiments, the formulation comprises 2.4 mg tri-potassium citrate, or an equivalent molar amount of a hydrate thereof.

In some embodiments, the formulation comprises 0.01 mg to 1.0 mg tri-potassium citrate, 0.05 mg to 0.5 mg tri-potassium citrate, 0.1 mg to 0.3 mg tri-potassium citrate, or 0.2 mg to 0.25 mg tri-potassium citrate, or an equivalent molar amount of a hydrate thereof. In certain embodiments, the formulation comprises 0.24 mg tri-potassium citrate, or an equivalent molar amount of a hydrate thereof.

In another aspect, provided herein is a pharmaceutical formulation suitable for oral mucosal (e.g., sublingual and/or buccal) administration comprising:
91 mg to 200 mg micronized aspirin;
citric acid anhydrous;
tri-potassium citrate monohydrate;
sodium lauryl sulfate; and
one or more pharmaceutically acceptable excipients.

In another aspect, provided herein is a pharmaceutical formulation suitable for oral mucosal (e.g., sublingual and/or buccal) administration comprising:
91 mg to 200 mg aspirin;
citric acid anhydrous;
tri-potassium citrate monohydrate;
sodium lauryl sulfate; and
one or more pharmaceutically acceptable excipients.

In some embodiments, the formulation comprises 91 mg to 100 mg micronized aspirin, 100 mg to 110 mg micronized aspirin, 110 mg to 120 mg micronized aspirin, 120 mg to 130 mg micronized aspirin, 130 mg to 140 mg micronized aspirin, 140 mg to 150 mg micronized aspirin, 150 mg to 160 mg micronized aspirin, 160 mg to 170 mg micronized aspirin, 170 mg to 180 mg micronized aspirin, 180 mg to 190 mg micronized aspirin, 190 mg to 200 mg micronized aspirin, 100 mg to 120 mg micronized aspirin, 120 mg to 140 mg micronized aspirin, 140 mg to 160 mg micronized aspirin, 160 mg to 180 mg micronized aspirin, 180 mg to 200 mg micronized aspirin, 150 mg to 170 mg micronized aspirin, 91 mg to 150 mg micronized aspirin, or 150 mg to 200 mg micronized aspirin. In some embodiments, the formulation comprises 100 mg micronized aspirin, 110 mg micronized aspirin, 120 mg micronized aspirin, 130 mg micronized aspirin, 140 mg micronized aspirin, 150 mg micronized aspirin, 160 mg micronized aspirin, 170 mg micronized aspirin, 180 mg micronized aspirin, 190 mg micronized aspirin, or 200 mg micronized aspirin. In some embodiments, the formulation comprises 100 mg to 200 mg micronized aspirin. In some embodiments, the formulation comprises 150 mg to 200 mg micronized aspirin.

In some embodiments, the formulation comprises 91 mg micronized aspirin, 95 mg micronized aspirin, 100 mg micronized aspirin, 105 mg micronized aspirin, 110 mg micronized aspirin, 115 mg micronized aspirin, 120 mg micronized aspirin, 125 mg micronized aspirin, 130 mg micronized aspirin, 135 mg micronized aspirin, 140 mg micronized aspirin, 145 mg micronized aspirin, 150 mg micronized aspirin, 155 mg micronized aspirin, 160 mg micronized aspirin, 165 mg micronized aspirin, 170 mg micronized aspirin, 175 mg micronized aspirin, 180 mg micronized aspirin, 185 mg micronized aspirin, 190 mg micronized aspirin, 195 mg micronized aspirin, or 200 mg micronized aspirin.

In some embodiments, the formulation comprises 100 mg to 163 mg micronized aspirin. In some embodiments, the formulation comprises 145 mg to 163 mg micronized aspirin. In certain embodiments, the formulation comprises 163 mg micronized aspirin. In certain embodiments, the formulation comprises 162.5 mg micronized aspirin.

In some embodiments, the formulation comprises 91 mg to 100 mg aspirin, 100 mg to 110 mg aspirin, 110 mg to 120 mg aspirin, 120 mg to 130 mg aspirin, 130 mg to 140 mg aspirin, 140 mg to 150 mg aspirin, 150 mg to 160 mg aspirin, 160 mg to 170 mg aspirin, 170 mg to 180 mg aspirin, 180 mg to 190 mg aspirin, 190 mg to 200 mg aspirin, 100 mg to 120 mg aspirin, 120 mg to 140 mg aspirin, 140 mg to 160 mg aspirin, 160 mg to 180 mg aspirin, 180 mg to 200 mg aspirin, 150 mg to 170 mg aspirin, 91 mg to 150 mg aspirin, or 150 mg to 200 mg aspirin. In some embodiments, the formulation comprises 100 mg aspirin, 110 mg aspirin, 120 mg aspirin, 130 mg aspirin, 140 mg aspirin, 150 mg aspirin, 160 mg aspirin, 170 mg aspirin, 180 mg aspirin, 190 mg aspirin, or 200 mg aspirin. In some embodiments, the formulation comprises 100 mg to 200 mg aspirin. In some embodiments, the formulation comprises 150 mg to 200 mg aspirin.

In some embodiments, the formulation comprises 91 mg aspirin, 95 mg aspirin, 100 mg aspirin, 105 mg aspirin, 110 mg aspirin, 115 mg aspirin, 120 mg aspirin, 125 mg aspirin, 130 mg aspirin, 135 mg aspirin, 140 mg aspirin, 145 mg aspirin, 150 mg aspirin, 155 mg aspirin, 160 mg aspirin, 165 mg aspirin, 170 mg aspirin, 175 mg aspirin, 180 mg aspirin, 185 mg aspirin, 190 mg aspirin, 195 mg aspirin, or 200 mg aspirin.

In some embodiments, the formulation comprises 100 mg to 163 mg aspirin. In some embodiments, the formulation comprises 145 mg to 163 mg aspirin. In certain embodiments, the formulation comprises 163 mg aspirin. In certain embodiments, the formulation comprises 162.5 mg aspirin.

In some embodiments, the formulation comprises granules comprising an intragranular fraction, wherein the intragranular fraction comprises the aspirin. In some embodiments, the intragranular fraction further comprises the citric acid anhydrous and the tri-potassium citrate monohydrate. In some embodiments, the intragranular fraction further comprises one or more pharmaceutically acceptable excipients (e.g., sodium lauryl sulfate and/or aspartame).

In some embodiments, the formulation does not comprise granules.

In some embodiments, the formulation comprises 8 mg to 75 mg citric acid anhydrous, 10 mg to 75 mg citric acid anhydrous, 15 mg to 75 mg citric acid anhydrous, 20 mg to 75 mg citric acid anhydrous, 25 mg to 75 mg citric acid anhydrous, 30 mg to 75 mg citric acid anhydrous, 35 mg to 75 mg citric acid anhydrous, 40 mg to 75 mg citric acid anhydrous, 45 mg to 75 mg citric acid anhydrous, 50 mg to 75 mg citric acid anhydrous, 55 mg to 75 mg citric acid anhydrous, 60 mg to 75 mg citric acid anhydrous, 8 mg to 70 mg citric acid anhydrous, 8 mg to 65 mg citric acid anhydrous, 8 mg to 60 mg citric acid anhydrous, 8 mg to 55 mg citric acid anhydrous, 8 mg to 50 mg citric acid anhydrous, 8 mg to 45 mg citric acid anhydrous, 8 mg to 40 mg citric acid anhydrous, 8 mg to 35 mg citric acid anhydrous, 8 mg to 30 mg citric acid anhydrous, 8 mg to 25 mg citric acid anhydrous, 8 mg to 20 mg citric acid anhydrous, 8 mg to 15 mg citric acid anhydrous, 10 mg to 70 mg citric acid anhydrous, 15 mg to 65 mg citric acid anhydrous, 20 mg to 60 mg citric acid anhydrous, 25 mg to 55 mg citric acid anhydrous, 30 mg to 50 mg citric acid anhydrous, or 35 mg to 45 mg citric acid anhydrous. In some embodiments, the formulation comprises 8 mg to 75 mg citric acid anhydrous. In some embodiments, the formulation comprises 8 mg to 15 mg citric acid anhydrous.

In some embodiments, the formulation comprises 8 mg citric acid anhydrous, 10 mg citric acid anhydrous, 15 mg citric acid anhydrous, 20 mg citric acid anhydrous, 25 mg citric acid anhydrous, 30 mg citric acid anhydrous, 35 mg citric acid anhydrous, 40 mg citric acid anhydrous, 45 mg citric acid anhydrous, 50 mg citric acid anhydrous, 55 mg citric acid anhydrous, 60 mg citric acid anhydrous, 65 mg citric acid anhydrous, 70 mg citric acid anhydrous, or 75 mg citric acid anhydrous. In certain embodiments, the formulation comprises 8.2 mg citric acid anhydrous.

In some embodiments, the formulation comprises 0.1 mg citric acid anhydrous, 0.2 mg citric acid anhydrous, 0.3 mg citric acid anhydrous, 0.4 mg citric acid anhydrous, 0.5 mg citric acid anhydrous, 0.6 mg citric acid anhydrous, 0.7 mg citric acid anhydrous, 0.8 mg citric acid anhydrous, 0.9 mg citric acid anhydrous, or 1.0 mg citric acid anhydrous. In certain embodiments, the formulation comprises 0.82 mg citric acid anhydrous.

In some embodiments, the formulation comprises 2.2 mg to 21 mg tri-potassium citrate monohydrate, 4 mg to 21 mg tri-potassium citrate monohydrate, 6 mg to 21 mg tri-potassium citrate monohydrate, 8 mg to 21 mg tri-potassium citrate monohydrate, 10 mg to 21 mg tri-potassium citrate monohydrate, 12 mg to 21 mg tri-potassium citrate monohydrate, 14 mg to 21 mg tri-potassium citrate monohydrate, 16 mg to 21 mg tri-potassium citrate monohydrate, 18 mg to 21 mg tri-potassium citrate monohydrate, 2.2 mg to 19 mg tri-potassium citrate monohydrate, 2.2 mg to 17 mg tri-potassium citrate monohydrate, 2.2 mg to 15 mg tri-potassium citrate monohydrate, 2.2 mg to 13 mg tri-potassium citrate monohydrate, 2.2 mg to 11 mg tri-potassium citrate monohydrate, 2.2 mg to 9 mg tri-potassium citrate monohydrate, 2.2 mg 7 mg tri-potassium citrate monohydrate, 2.2 mg to 5 mg tri-potassium citrate monohydrate, 2 mg to 21 mg tri-potassium citrate monohydrate, 4 mg to 19 mg tri-potassium citrate monohydrate, 6 mg to 17 mg tri-potassium citrate monohydrate, 8 mg to 15 mg tri-potassium citrate monohydrate, or 10 mg to 13 mg tri-potassium citrate monohydrate. In some embodiments, the formulation comprises 2.2 mg to 21 mg tri-potassium citrate monohydrate. In some embodiments, the formulation comprises 0.3 mg to 2.9 mg tri-potassium citrate monohydrate.

In some embodiments, the formulation comprises 2.2 mg tri-potassium citrate monohydrate, 3 mg tri-potassium citrate monohydrate, 5 mg tri-potassium citrate monohydrate, 7 mg tri-potassium citrate monohydrate, 9 mg tri-potassium citrate monohydrate, 11 mg tri-potassium citrate monohydrate, 13 mg tri-potassium citrate monohydrate, 15 mg tri-potassium citrate monohydrate, 17 mg tri-potassium citrate monohydrate, 19 mg tri-potassium citrate monohydrate, or 21 mg tri-potassium citrate monohydrate. In certain embodiments, the formulation comprises 2.4 mg tri-potassium citrate monohydrate.

In some embodiments, the formulation comprises 0.01 mg to 1.0 mg tri-potassium citrate monohydrate, 0.05 mg to 0.5 mg tri-potassium citrate monohydrate, 0.1 mg to 0.3 mg tri-potassium citrate monohydrate, or 0.2 mg to 0.25 mg tri-potassium citrate monohydrate. In certain embodiments, the formulation comprises 0.24 mg tri-potassium citrate monohydrate.

In some embodiments, the citric acid anhydrous and tri-potassium citrate monohydrate are present in amounts of 3 parts to 26 parts citric acid anhydrous per one part tri-potassium citrate monohydrate by weight. In some embodiments, the citric acid anhydrous and tri-potassium citrate monohydrate are present in amounts of 3.6 parts to 26 parts citric acid anhydrous per one part tri-potassium citrate monohydrate by weight. In some embodiments, the citric acid anhydrous and tri-potassium citrate monohydrate are present in amounts of 3 parts, 4 parts, 5 parts, 6 parts, 7 parts, 8 parts, 9 parts, 10 parts, 11 parts, 12 parts, 13 parts, 14 parts, 15 parts, 16 parts, 17 parts, 18 parts, 19 parts, 20 parts, or 21 parts citric acid anhydrous per one part tri-potassium citrate monohydrate by weight.

In some embodiments, the citric acid anhydrous and tri-potassium citrate monohydrate are present in amounts of 3.4 parts to 26 parts citric acid anhydrous per one part tri-potassium citrate monohydrate by weight.

In another aspect, the formulation comprises 3 mg to 12 mg sodium lauryl sulfate, 4 mg to 12 mg sodium lauryl sulfate, 5 mg to 12 mg sodium lauryl sulfate, 6 mg to 12 mg sodium lauryl sulfate, 7 mg to 12 mg sodium lauryl sulfate, 8 mg to 12 mg sodium lauryl sulfate, 9 mg to 12 mg sodium lauryl sulfate, 10 mg to 12 mg sodium lauryl sulfate, 3 mg to 11 mg sodium lauryl sulfate, 3 mg to 10 mg sodium lauryl sulfate, 3 mg to 9 mg sodium lauryl sulfate, 3 mg to 8 mg sodium lauryl sulfate, 3 mg to 7 mg sodium lauryl sulfate, 3 mg to 6 mg sodium lauryl sulfate, 3 mg to 5 mg sodium lauryl sulfate, 3 mg to 12 mg sodium lauryl sulfate, 4 mg to 11 mg sodium lauryl sulfate, 5 mg to 10 mg sodium lauryl sulfate, or 6 mg to 9 mg sodium lauryl sulfate. In some embodiments, the formulation comprises 3 mg to 12 mg sodium lauryl sulfate. In some embodiments, the formulation comprises 3 mg to 6 mg sodium lauryl sulfate.

In some embodiments, the formulation comprises 3 mg sodium lauryl sulfate, 4 mg sodium lauryl sulfate, 5 mg sodium lauryl sulfate, 6 mg sodium lauryl sulfate, 7 mg sodium lauryl sulfate, 8 mg sodium lauryl sulfate, 9 mg sodium lauryl sulfate, 10 mg sodium lauryl sulfate, 11 mg sodium lauryl sulfate, or 12 mg sodium lauryl sulfate. In certain embodiments, the formulation comprises 3.7 mg sodium lauryl sulfate.

In another aspect, provided herein is a pharmaceutical formulation suitable for oral mucosal (e.g., sublingual and/or buccal) administration comprising:
162.5 mg micronized aspirin;
8.2 mg citric acid anhydrous;
2.4 mg tri-potassium citrate monohydrate;
3.7 mg sodium lauryl sulfate; and
one or more pharmaceutically acceptable excipients.

In another aspect, provided herein is a pharmaceutical formulation suitable for oral mucosal (e.g., sublingual and/or buccal) administration comprising:
162.5 mg micronized aspirin;
0.82 mg citric acid anhydrous;
0.24 mg tri-potassium citrate monohydrate;
3.65 mg sodium lauryl sulfate; and
one or more pharmaceutically acceptable excipients.

In another aspect, provided herein is a pharmaceutical formulation suitable for oral mucosal (e.g., sublingual and/or buccal) administration comprising:
162.5 mg aspirin;
8.2 mg citric acid anhydrous;
2.4 mg tri-potassium citrate monohydrate;
3.7 mg sodium lauryl sulfate; and
one or more pharmaceutically acceptable excipients.

In another aspect, provided herein is a pharmaceutical formulation suitable for oral mucosal (e.g., sublingual and/or buccal) administration comprising:
162.5 mg aspirin;
0.82 mg citric acid anhydrous;
0.24 mg tri-potassium citrate monohydrate;
3.65 mg sodium lauryl sulfate; and
one or more pharmaceutically acceptable excipients.

In some embodiments, the formulation comprises granules comprising an intragranular fraction, wherein the intragranular fraction comprises the aspirin. In some embodiments, the intragranular fraction further comprises the citric acid anhydrous and the tri-potassium citrate monohydrate. In some embodiments, the intragranular fraction further comprises one or more pharmaceutically acceptable excipients (e.g., sodium lauryl sulfate and/or aspartame).

In some embodiments, the formulation does not comprise granules.

In some embodiments, the formulations disclosed herein comprise an anti-settling and/or anti-sagging agent. In certain embodiments, the anti-settling and/or anti-sagging agent is fumed silica.

In some embodiments, the formulations disclosed herein comprise 6 mg to 30 mg fumed silica, 10 mg to 30 mg fumed silica, 14 mg to 30 mg fumed silica, 18 mg to 30 mg fumed silica, 22 mg to 30 mg fumed silica, 26 mg to 30 mg fumed silica, 6 mg to 26 mg fumed silica, 6 mg to 22 mg fumed silica, 6 mg to 18 mg fumed silica, 6 mg to 14 mg fumed silica, 6 mg to 10 mg fumed silica, 10 mg to 26 mg fumed silica, or 14 mg to 22 mg fumed silica. In some embodiments, the formulations disclosed herein comprise 6 mg to 30 mg fumed silica. In some embodiments, the formulations disclosed herein comprise 6 mg to 10 mg fumed silica.

In some embodiments, the formulations disclosed herein comprise 6 mg fumed silica, 8 mg fumed silica, 10 mg fumed silica, 12 mg fumed silica, 14 mg fumed silica, 16 mg fumed silica, 18 mg fumed silica, 20 mg fumed silica, 22 mg fumed silica, 24 mg fumed silica, 26 mg fumed silica, 28 mg fumed silica, or 30 mg fumed silica. In certain embodiments, the formulations disclosed herein comprise 8.7 mg fumed silica.

In some embodiments, the anti-settling and/or anti-sagging agent is 4% to 10%, 5% to 10%, 6% to 10%, 7% to 10%, 8% to 10%, 9% to 10%, 4% to 9%, 4% to 8%, 4% to 7%, 4% to 6%, 4% to 5%, 4% to 9%, or 5% to 8% of the formulation by mass. In certain embodiments, the anti-settling and/or anti-sagging agent is 4% to 10% of the formulation by mass. In some embodiments, the anti-settling and/or anti-sagging agent is 4%, 5%, 6%, 7%, 8%, 9%, or 10% of the formulation by mass. In certain embodiments, the anti-settling and/or anti-sagging agent is 4.7% of the formulation by mass.

In some embodiments, the formulations disclosed herein comprise a sweetening agent. In certain embodiments, the sweetening agent is aspartame.

In some embodiments, the formulations disclosed herein comprise 6 mg to 30 mg aspartame, 10 mg to 30 mg aspartame, 14 mg to 30 mg aspartame, 18 mg to 30 mg aspartame, 22 mg to 30 mg aspartame, 26 mg to 30 mg aspartame, 6 mg to 26 mg aspartame, 6 mg to 22 mg aspartame, 6 mg to 18 mg aspartame, 6 mg to 14 mg aspartame, 6 mg to 10 mg aspartame, 10 mg to 26 mg aspartame, or 14 mg to 22 mg aspartame. In some embodiments, the formulations disclosed herein comprise 6 mg to 30 mg aspartame. In some embodiments, the formulations disclosed herein comprise 6 mg to 10 mg aspartame.

In some embodiments, the formulations disclosed herein comprise 6 mg aspartame, 8 mg aspartame, 10 mg aspartame, 12 mg aspartame, 14 mg aspartame, 16 mg aspartame, 18 mg aspartame, 20 mg aspartame, 22 mg aspartame, 24 mg aspartame, 26 mg aspartame, 28 mg aspartame, or 30 mg aspartame. In certain embodiments, the formulations disclosed herein comprise 8.7 mg aspartame.

In some embodiments, the formulation comprises one or both of aspartame and fumed silica.

In some embodiments, at least 50% of the aspirin is released (i.e., dissolved) within 2 minutes using a paddle dissolution apparatus (e.g., a USP <711> Type 2 dissolution apparatus, 500 mL pH 6.8 potassium phosphate buffer, paddle speed 50 rpm, 37° C.). In some embodiments, at least 55% of the aspirin is released within 2 minutes using a paddle dissolution apparatus (e.g., a USP <711> Type 2 dissolution apparatus, 500 mL pH 6.8 potassium phosphate buffer, paddle speed 50 rpm, 37° C.). In some embodiments, at least 60% of the aspirin is released within 2 minutes using a paddle dissolution apparatus (e.g., a USP <711> Type 2 dissolution apparatus, 500 mL pH 6.8 potassium phosphate buffer, paddle speed 50 rpm, 37° C.). In some embodiments, at least 65% of the aspirin is released within 2 minutes using a paddle dissolution apparatus (e.g., a USP <711> Type 2 dissolution apparatus, 500 mL pH 6.8 potassium phosphate buffer, paddle speed 50 rpm, 37° C.). In some embodiments, at least 70% of the aspirin is released within 2 minutes using a paddle dissolution apparatus (e.g., a USP <711> Type 2 dissolution apparatus, 500 mL pH 6.8 potassium phosphate buffer, paddle speed 50 rpm, 37° C.). In some embodiments, at least 75% of the aspirin is released within 2 minutes using a paddle dissolution apparatus (e.g., a USP <711> Type 2 dissolution apparatus, 500 mL pH 6.8 potassium phosphate buffer, paddle speed 50 rpm, 37° C.). In some embodiments, at least 80% of the aspirin is released within 2 minutes using a paddle dissolution apparatus (e.g., a USP <711> Type 2 dissolution apparatus, 500 mL pH 6.8 potassium phosphate buffer, paddle speed 50 rpm, 37° C.). In some embodiments, at least 85% of the aspirin is released within 2 minutes using a paddle dissolution apparatus (e.g., a USP <711> Type 2 dissolution apparatus, 500 mL pH 6.8 potassium phosphate buffer, paddle speed 50 rpm, 37° C.). In some embodiments, at least 50% of the aspirin is released within 5 minutes using a paddle dissolution apparatus (e.g., a USP <711> Type 2 dissolution apparatus, 500 mL pH 6.8 potassium phosphate buffer, paddle speed 50 rpm, 37° C.). In some embodiments, at least 55% of the aspirin is released within 5 minutes using a paddle dissolution apparatus (e.g., a USP <711> Type 2 dissolution apparatus, 500 mL pH 6.8 potassium phosphate buffer, paddle speed 50 rpm, 37° C.). In some embodiments, at least 60% of the aspirin is released within 5 minutes using a paddle dissolution apparatus (e.g., a USP <711> Type 2 dissolution apparatus, 500 mL pH 6.8 potassium phosphate buffer, paddle speed 50 rpm, 37° C.). In some embodiments, at least 65% of the aspirin is released within 5 minutes using a paddle dissolution apparatus (e.g., a USP <711> Type 2 dissolution apparatus, 500 mL pH 6.8 potassium phosphate buffer, paddle speed 50 rpm, 37° C.). In some embodiments, at least 70% of the aspirin is released within 5 minutes using a paddle dissolution apparatus (e.g., a USP <711> Type 2 dissolution apparatus, 500 mL pH 6.8 potassium phosphate buffer, paddle speed 50 rpm, 37° C.). In some embodiments, at least 75% of the aspirin is released within 5 minutes using a paddle dissolution apparatus (e.g., a USP <711> Type 2 dissolution apparatus, 500 mL pH 6.8 potassium phosphate buffer, paddle speed 50 rpm, 37° C.). In some embodiments, at least 80% of the aspirin is released within 5 minutes using a paddle dissolution apparatus (e.g., a USP <711> Type 2 dissolution apparatus, 500 mL pH 6.8 potassium phosphate buffer, paddle speed 50 rpm, 37° C.). In some embodiments, at least 85% of the aspirin is released within 5 minutes using a paddle dissolution apparatus (e.g., a USP <711> Type 2 dissolution apparatus, 500 mL pH 6.8 potassium phosphate buffer, paddle speed 50 rpm, 37° C.).

In some embodiments, at least 70% of the aspirin is released within 10 minutes using a paddle dissolution apparatus (e.g., a USP <711> Type 2 dissolution apparatus, 500 mL pH 6.8 potassium phosphate buffer, paddle speed 50 rpm, 37° C.). In some embodiments, at least 75% of the aspirin is released within 10 minutes using a paddle dissolution apparatus (e.g., a USP <711> Type 2 dissolution apparatus, 500 mL pH 6.8 potassium phosphate buffer, paddle speed 50 rpm, 37° C.). In some embodiments, at least 80% of the aspirin is released within 10 minutes using a paddle dissolution apparatus (e.g., a USP <711> Type 2 dissolution apparatus, 500 mL pH 6.8 potassium phosphate buffer, paddle speed 50 rpm, 37° C.). In some embodiments, at least 85% of the aspirin is released within 10 minutes using a paddle dissolution apparatus (e.g., a USP <711> Type 2 dissolution apparatus, 500 mL pH 6.8 potassium phosphate buffer, paddle speed 50 rpm, 37° C.).

In some embodiments, at least 80% of the aspirin is released within 30 minutes using a paddle dissolution apparatus (e.g., a USP <711> Type 2 dissolution apparatus, 500 mL pH 6.8 potassium phosphate buffer, paddle speed 50 rpm, 37° C.). In some embodiments, at least 85% of the aspirin is released within 30 minutes using a paddle dissolution apparatus (e.g., a USP <711> Type 2 dissolution apparatus, 500 mL pH 6.8 potassium phosphate buffer, paddle speed 50 rpm, 37° C.). In some embodiments, at least 90% of the aspirin is released within 30 minutes using a paddle dissolution apparatus (e.g., a USP <711> Type 2 dissolution apparatus, 500 mL pH 6.8 potassium phosphate buffer, paddle speed 50 rpm, 37° C.). In some embodiments, at least 95% of the aspirin is released within 30 minutes using a paddle dissolution apparatus (e.g., a USP <711> Type 2 dissolution apparatus, 500 mL pH 6.8 potassium phosphate buffer, paddle speed 50 rpm, 37° C.).

Determination of the dissolution rate of a formulation disclosed herein may be accomplished by methods known in the art and as described herein, e.g., using a USP <711> Type 2 dissolution apparatus, 500 mL pH 6.8 potassium phosphate buffer, paddle speed 50 rpm, 37° C.

In some embodiments, the formulation is a solid dosage form. In some embodiments, the formulation is in powder form. In some embodiments, the formulation is in tablet form. In some embodiments, the tablet is an orally disintegrating tablet.

In another aspect, provided herein is a pharmaceutical formulation suitable for oral mucosal (e.g., sublingual and/or buccal) administration comprising:
91 mg to 200 mg micronized aspirin;
a buffer;
a surfactant;
and one or more pharmaceutically acceptable excipients,
wherein the pH of a solution of the formulation dissolved in 1 ml distilled water is less than or equal to 3, wherein the formulation is manufactured using a process comprising:
   mixing the aspirin with one or more pharmaceutically acceptable excipients to form a mixture;
   micronizing the mixture to form a micronized aspirin pre-blend;
   wet granulating the micronized aspirin pre-blend with a granulation fluid to form a granulated composition; and
   blending the granulated composition with one or more pharmaceutically acceptable excipients to afford the formulation.

In some embodiments, the aspirin is mixed with at least citric acid anhydrous, tri-potassium citrate monohydrate, and a surfactant. In certain embodiments, the surfactant is an anionic surfactant. In certain embodiments, the surfactant is sodium lauryl sulfate. In some embodiments, mixing the aspirin with the pharmaceutically acceptable excipients prior to micronization achieves improved uniform distribution of aspirin in the blend.

In some embodiments, the granulation fluid is aqueous. In some embodiments, the granulation fluid comprises a surfactant. In certain embodiments, the surfactant is an anionic surfactant. In certain embodiments, the surfactant is sodium lauryl sulfate.

In some embodiments, the granulated composition is blended with an anti-settling and/or anti-sagging agent. In some embodiments, the anti-settling agent is fumed silica.

In another aspect, provided herein is a pharmaceutical formulation suitable for oral mucosal (e.g., sublingual and/or buccal) administration comprising:
91 mg to 200 mg micronized aspirin;
a buffer;
a surfactant;
and one or more pharmaceutically acceptable excipients,
wherein the pH of a solution of the formulation dissolved in 1 ml distilled water is less than or equal to 3, wherein the formulation is manufactured using a process comprising:
   mixing the aspirin with one or more pharmaceutically acceptable excipients to form a mixture;
   micronizing the mixture to form a micronized aspirin dry blend; and
   blending the micronized aspirin dry blend with one or more pharmaceutically acceptable excipients to afford the formulation.

In some embodiments, the aspirin is mixed with at least citric acid anhydrous, tri-potassium citrate monohydrate, and a surfactant. In certain embodiments, the surfactant is an anionic surfactant. In certain embodiments, the surfactant is sodium lauryl sulfate. In some embodiments, mixing the aspirin with the pharmaceutically acceptable excipients prior to micronization achieves improved uniform distribution of aspirin in the blend.

In some embodiments, the micronized aspirin dry blend is blended with an anti-settling and/or anti-sagging agent. In some embodiments, the anti-settling and/or anti-sagging agent is fumed silica.

### Routes of Administration

The pharmaceutical formulations described herein can be administered via oral mucosal delivery. In an embodiment, the pharmaceutical formulations described herein are administered to a subject in need thereof in the form of a powder. In an embodiment, the pharmaceutical formulations described herein are administered to a subject in need thereof in the form of a tablet. In some embodiments, the pharmaceutical formulations described herein are administered supralingually. In some embodiments, the pharmaceutical formulations described herein are administered sublingually. In some embodiments, the pharmaceutical formulations described herein are administered buccally.

### Methods of Treatment

In one aspect, provided herein are methods of treating a disease, disorder, or condition, or reducing the symptoms of said disease, disorder, or condition, comprising administering to a subject in need thereof one or more pharmaceutical formulations described herein. In some embodiments, the disease, disorder, or condition is myocardial infarction. In some embodiments, the disease, disorder, or condition is selected from pain, fever, and swelling. In some embodiments, the disease, disorder, or condition is a bacterial or viral infection, and the method reduces the symptoms of the bacterial or viral infection. In some embodiments, the method provides an anti-platelet effect. In some embodiments, the method provides an anti-inflammatory effect. In some embodiments, the method provides an anti-pyretic effect. In some embodiments, the method provides an anti-thrombotic effect.

In some embodiments, the methods provided herein comprise oral-mucosally (e.g., sublingually and/or buccally) administering to the subject one or more pharmaceutical formulations described herein.

Contemplated methods may include administering to the subject one or more pharmaceutical formulations described herein once per week, twice per week, three times per week, four times per week, five times per week, six times per week, once a day, twice a day, three times a day, four times a day, or five times a day. In some embodiments, a pharmaceutical formulation described herein may be administered once per day. In some embodiments, a formulation may be administered for 1 minute, 2 minutes, 3 minutes, 4 minutes, 5 minutes, 6 minutes, 7 minutes, 8 minutes, 9 minutes, or 10 minutes.

In some embodiments, the methods provided herein comprise administering to the subject one formulation described herein. In some embodiments, the methods provided herein comprise concurrently administering to the subject two formulations described herein. In some embodiments, the methods provided herein comprise concurrently administering to the subject three formulations described herein. In some embodiments, the methods provided herein comprise concurrently administering to the subject four formulations described herein.

### Kits

In one aspect, the disclosure provides a kit for the treatment of a disease, disorder, or condition, or reducing the symptoms of said disease, disorder, or condition, described herein, comprising one or more pharmaceutical formulations described herein.

In one aspect, provided herein is a kit for suitable storage of one or more pharmaceutical formulation described herein. In some embodiments, the kit comprises a pouch suitable for packaging any of the pharmaceutical formulations provided herein.

Provided herein, in an embodiment, is a product including one or more pharmaceutical formulation described herein contained in a kit. Contemplated kits may include a container. In some embodiments, the kit comprises a primary container and a secondary container. In some embodiments, the primary container is a pouch. In some embodiments, the pouch comprises a single pharmaceutical formulation disclosed herein. In some embodiments, the single dose of a pharmaceutical formulation is a powder for oral mucosal (e.g., sublingual and/or buccal delivery). In some embodiments, the secondary container is a box. In some embodiments, a box contains 1, 2, 3, 4, 5, 6, 7, 8, 9, or 10 pouched powder dosage forms for oral mucosal (e.g., sublingual and/or buccal) delivery. In some aspects, contemplated kits may comprise instructions for use in treating a disease, disorder, or condition described herein and/or provide instructions for storage, e.g., instructions to store between 15° C. to 30° C.

### EXEMPLIFICATION

The representative examples that follow are intended to help illustrate the disclosure, and are not intended to, nor should they be construed to, limit the scope of the disclosure.

### Abbreviations

API active pharmaceutical ingredient
BU blend uniformity
CU content uniformity
FB/FBG fluid bed/fluid bed granulation
HPLC high performance liquid chromatography
RPM/rpm revolutions per minute
SLS sodium lauryl sulfate
T₀ beginning time
T_{end} end time
USP United States Pharmacopoeia

### Example 1. Granulation and Blending Processes for Blending of Oral Mucosal Aspirin Formulations

This example describes micronization and granulation processes useful for manufacturing aspirin formulations for oral mucosal (e.g., sublingual and/or buccal) delivery. An exemplary formulation for oral-mucosal (e.g., sublingual or buccal) delivery is provided in Table 1.

TABLE 1
Exemplary Formulation Composition (100 g)

| | | | | Amount |
|---|---|---|---|---|
| | | Amount | % | per dose |
| Ingredients | Function | (g) | w/w | (mg) |
| Aspirin USP 80 mesh | API | 83.72 | 83.72 | 162.5 |
| Powder | | | | |
| Citric Acid | pH adjustment | 4.22 | 4.22 | 8.2 |
| anhydrous, powder | conjugate acid | | | |
| | (buffer reagent) | | | |
| Tri-potassium citrate | conjugate base | 1.24 | 1.24 | 2.4 |
| monohydrate | (buffer reagent) | | | |
| Aspartame 100 mesh | taste masking | 4.47 | 4.47 | 8.68 |
| powder | agent | | | |
| Sodium Lauryl | Surfactant or | 1.88 | 1.88 | 3.65 |
| Sulfate | detergent/wetting | | | |
| | agent | | | |
| Aerosil 200 Pharma | anti-settling/anti- | 4.47 | 4.47 | 8.68 |
| | sagging agent | | | |
| Total | | 100.00 | 100.00 | 194.11 |

API and excipients are obtained from reputable commercial sources and are USP quality controlled.

An oral mucosal (e.g., sublingual and/or buccal) aspirin formulation is prepared using a process comprising fluid bed granulation. Aspirin USP 80 mesh powder, tri-potassium citrate monohydrate, and aspartame 100 mesh powder are sieved using a Porta Sifter screener. The resulting mixture is blended with citric acid anhydrous powder and sodium lauryl sulfate using a V blender, and the blended mixture is micronized using a jet mill micronizer (Sturtevant Micronizer) to afford a micronized aspirin pre-blend. The micronized aspirin pre-blend is granulated using top spray granulation in a Glatt GPCG 3.1 fluid bed dryer, using a granulation fluid containing purified water USP and 0.3% sodium lauryl sulfate to afford a granulated composition. The granulated composition is then sieved with Aerosil 200 Pharma using Hand Screen U.S. Std Mesh #20, and the resulting mixture is blended using a bin blender to afford a granulated powder formulation for sublingual and/or buccal delivery.

An oral mucosal (e.g., sublingual and/or buccal) aspirin formulation is alternatively prepared using a process comprising dry blending. Aspirin USP 80 mesh powder, tri-potassium citrate monohydrate, and aspartame 100 mesh powder are sieved using a Porta Sifter. The resulting mixture is blended with citric acid anhydrous powder and sodium lauryl sulfate using a V Blender, and the blended mixture is micronized using a jet mill micronizer (Sturtevant Micronizer) to afford a micronized aspirin dry blend. Aerosil 200 Pharma is sieved using Hand Screen U.S. Std Mesh #20, and the resulting sieved Aerosil 200 Pharma is blended with the micronized aspirin dry blend using a bin blender to afford a powder formulation for sublingual delivery.

In each formulation method (i.e., fluid bed granulation and dry blending), micronization of the aspirin together with additional excipients (e.g., tri-potassium citrate monohydrate, citric acid anhydrous, and sodium lauryl sulfate) is expected to improve uniform distribution of aspirin in the blend. The blend uniformity is expected to be maintained through subsequent process steps.

### Preparation of Pre-Blend Batch Using Air Jet Milling

A micronized aspirin pre-blend is prepared by air jet milling as described above with an exemplary batch size of 5.5 kg. To remove large lumps and prevent interference with the feed rate, individual ingredients are screened through US mesh #14. Exemplary formulation composition and process parameters for preparation of the pre-blend are provided in Table 2 and Table 3, respectively.

TABLE 2
Formulation Composition

| Ingredients | Amount (g) |
|---|---|
| Aspirin USP 80 mesh Powder | 4801 |
| Citric Acid anhydrous, powder | 242 |
| Tri-potassium citrate monohydrate | 71 |
| Vitasweet Aspartame 100 mesh | 256 |
| powder | |
| Sodium Lauryl Sulfate | 107 |

TABLE 3
Process Parameters

| Process Parameters | Data | |
|---|---|---|
| Bin Blender (CML-1200/35 L) RPM | 20 | |
| Blending Duration | 3 | min |
| Powder feed rate/Feeder setting | 485/425 | |
| Grind air/Feed air pressure | 50/50 | PSIG |
| Milling duration | 20 | min |

The pre-blend batch is then granulated using either top spray granulation or dry blending.

### Preparation of Fluid Bed Granulation Batch Using Top Spray Process

A fluid bed granulation batch is prepared using a top-spray process in a Glatt GPCG 3.1 fluid bed dryer with an exemplary batch size of 2.9 kg. Sodium lauryl sulfate (SLS) at 0.3% concentration in purified water USP (e.g., 267 mL) is sprayed on micronized aspirin pre-blend (e.g., 2.69 kg) during granulation. The resultant aspirin FB granules are blended with Aerosil 200 in an exemplary amount of 5.3 parts Aerosil 200 to 100 parts aspirin in the pre-blend by weight to obtain finished granular product. Exemplary process parameters for fluid bed granulation batch are provided in Table 4.

TABLE 4
Process Parameters

| Process Parameters | Data | |
|---|---|---|
| Product Temperature T₀-T_{end} | 24.1-35.7° | C. |
| Air Flow T₀-T_{end} | 32-44 | CFM |
| Spray rate T₀-T_{end} | 8-15 | g/min |
| Spraying Duration | ~27-40 | min |
| Bin Blender (CML-10/10 L) RPM | 18-20 | |
| Blending Duration | 3 | min |

### Preparation of Dry Blend Batch

A dry blend batch is prepared by blending a pre-blend batch (e.g., 2.66 kg) with Aerosil 200 (e.g., in an exemplary amount of 5.3 parts Aerosil 200 to 100 parts aspirin in the pre-blend by weight) in a CM 1200 bin blender with an exemplary batch size of 2.7 kg. Exemplary process parameters for dry blend granulation batch are provided in Table 5.

TABLE 5
Process Parameters

| Process Parameters | Data | |
|---|---|---|
| Bin Blender (CM-1200/35 L) RPM | 18 | rpm |
| Blending Duration | 3 | min |

### Aspirin Content Assay

The formulations may be analyzed for aspirin content using standard methods known in the art. Samples may be collected from each formulation using a 1.0 mm die, and the aspirin content for each batch may be analyzed by HPLC (exemplary methods provided below).

### HPLC Methods and Parameters

Standard preparation: Approximately 25 mg USP aspirin is transferred into a 25 mL amber volumetric flask and dissolved in 60/40 0.2% phosphoric acid in USP water/acetonitrile diluent to volume. The resulting solution is mixed well to afford a stock solution, which is diluted to a concentration of 0.1 mg/mL.

Sample preparation: formulation amounts equivalent to 200 mg aspirin are transferred into 100 mL volumetric flasks and dissolved in 0.2% phosphoric acid in 60/40 USP water/acetonitrile diluent to volume. The resulting solution is mixed well to afford a stock solution, which is diluted to a concentration of 0.1 mg/mL. The sample solutions are immediately transferred into HPLC vials and maintained in the HPLC system at 4° C.

Exemplary HPLC Instrument Details: Waters HPLC; Column: Hypersil GOLD aQ 4.6*250 mm, 5 micron; Column temperature: 30° C.; Sample temperature: 4° C.; Injection volume: 10 μL; Mobile Phase A: 0.2% phosphoric acid in USP water; Mobile Phase B: 0.2 phosphoric acid in ACN; Mobile phase gradient details: 100% A 0-15 min, 40/60 A/B 15-15.5 min, 100% B 15.5-19 min; Flow rate: 1.0 ml/min; Run time: 19 min; UV Detection Method: PDA, Wavelength: 215 nm

The aspirin content for each formulation is expected to meet or exceed 97% of expected aspirin content.

### Blend Uniformity

Top spray granulation and dry blend formulations may be analyzed for blend/content uniformity. In an exemplary procedure, ten blend uniformity (BU) samples are collected using a 1.0 mm die. Blend samples are prepared at concentrations of approximately 0.1 mg/mL. The samples are analyzed by HPLC using the same methods described above for the aspirin content assay.

The mean blend uniformity for each formulation is expected to meet or exceed 97%.

### Example 2. Determination of pH for Oral Mucosal (e.g., Sublingual and/or Buccal) Aspirin Formulations

The pH values of oral mucosal aspirin formulations may be determined using standard protocols as described in the United States Pharmacopoeia, Chapter <791>. In an exemplary procedure, USP water is added to each aspirin formulation, and the mixture is stirred for 15 minutes. The pH of the mixture is measured using a Mettler Toledo pH meter.

The pH values of the formulations described herein are expected to be about 2.5 to 3.5.

### Example 3. Dissolution of Oral Mucosal (e.g., Sublingual and/or Buccal) Aspirin Formulations

This example describes an exemplary procedure to determine the dissolution rate and solubility of formulations described herein.

### Dissolution Rate

Formulations are evaluated for their dissolution profiles. Samples are subjected to dissolution conditions in a Distek dissolution apparatus and are then analyzed by HPLC (dissolution and HPLC methods and parameters provided below):

### Dissolution Apparatus Conditions and Parameters

Apparatus: Distek dissolution bath coupled to Waters HPLC
Dissolution medium: pH 6.8 potassium phosphate buffer, 500 mL
Apparatus paddle speed: 50 rpm
n=3
Sample collection time points: 2, 6, 10, 15, 20 and 30 min
Sample collection volume: 9 mL
Temperature: 37° C.
Sample size: 1 dose equivalent (˜185.57 mg of formulation)

### HPLC Methods and Parameters

Standard preparation: Approximately 20 mg USP aspirin is diluted to volume with pH 6.8 phosphate buffer in 100 mL amber volumetric flask (the API is sonicated for 5 minutes in 70 mL pH 6.8 phosphate buffer first). The resulting solution is expected to have a concentration of 0.16 mg/mL aspirin API.

HPLC Instrument Details: Waters HPLC; Column: Hypersil GOLD aQ 4.6×250 mm, 5 micron; Column temperature: 30° C.; Sample temperature: 4° C.; Injection volume: 10 μL; Mobile Phase A: 0.2% phosphoric acid in USP water (ACN 50:50); Mobile Phase B: 0.2 phosphoric acid in ACN; Flow rate: 1.0 ml/min; Run time: 6 min; UV Detection Method: PDA, Wavelength: 280 nm

The formulations disclosed herein are expected to release at least 70% of their aspirin content within 6 minutes.

### Example 4. Granulation and Blending Processes for Blending of Oral Mucosal Aspirin Formulations

This example describes micronization and granulation processes useful for manufacturing aspirin formulations for oral mucosa (e. g., sublingual and/or buccal) delivery. Treatment A and Treatment B were prepared using the procedure described in this Example and their formulations are provided in Table 6 and Table 7, respectively.

TABLE 6
Formulation for Treatment A (Flavored Granules)

| | Amount (g) | % w/w |
|---|---|---|
| Aspirin USP | 162.5 g | 87.57% |
| Citric Acid | 0.82 g | 0.45% |
| Anhydrous | | |
| Potassium Citrate | 0.24 g | 0.13% |
| Monohydrate | | |
| Aspartame 100 mesh | 8.68 g | 4.68% |
| powder | | |
| Sodium Lauryl | 3.65 g | 1.95% |
| Sulfate | | |
| Aerosil 200 Pharma | 8.68 g | 4.68% |
| Lemon-Lime Natural | 1.00 g | 0.53% |
| Type Flavor | | |
| TOTAL | 185.57 g | 100% |

TABLE 7
Formulation for Treatment B (flavored powder)

| | Amount (g) | % w/w |
|---|---|---|
| Aspirin USP | 162.5 g | 87.57% |
| Citric Acid | 0.82 g | 0.45% |
| Anhydrous | | |
| Potassium Citrate | 0.24 g | 0.13% |
| Monohydrate | | |
| Aspartame 100 mesh | 8.68 g | 4.68% |
| powder | | |
| Sodium Lauryl | 3.65 g | 1.95% |
| Sulfate | | |
| Aerosil 200 Pharma | 8.68 g | 4.68% |
| Natural Orange | 1.00 g | 0.53% |
| Flavor WONF | | |
| TOTAL | 185.57 g | 100% |

API and excipients were obtained from reputable commercial sources and were USP quality controlled.

An oral mucosal (e.g., sublingual and/or buccal) aspirin formulation was prepared using a process comprising fluid bed granulation. Aspirin USP 80 mesh powder, citric acid anhydrous fine granular powder, granular tri-potassium citrate monohydrate, aspartame 100 mesh powder, and sodium lauryl sulfate were sieved using a Porta Sifter screener. The resulting mixture was blended using a 35-liter CM-1200 bin blender, and the blended mixture was micronized using a jet mill micronizer (Sturtevant Micronizer) to afford a micronized aspirin pre-blend. The micronized aspirin pre-blend was granulated using top spray granulation in a Glatt GPCG 3.1 fluid bed dryer, using a granulation fluid containing purified water USP and 0.3% sodium lauryl sulfate to afford a granulated composition. The granulated composition was then sieved with Aerosil 200 Pharma and Natural Orange Flavor WONF or Lemon-Lime Natural Type Flavor using Hand Screen U.S. Std Mesh #30, and the resulting mixture was blended using a 35-liter bin blender to afford a granulated powder formulation for sublingual and/or buccal delivery.

An oral mucosal (e.g., sublingual and/or buccal) aspirin formulation was alternatively prepared using a process comprising dry blending. Aspirin USP 80 mesh powder, citric acid anhydrous fine granular powder, granular tri-potassium citrate monohydrate, aspartame 100 mesh powder, and sodium lauryl sulfate were sieved using a Porta Sifter. The resulting mixture was blended using a V Blender, and the blended mixture was micronized using a jet mill micronizer (Sturtevant Micronizer) to afford a micronized aspirin dry blend. The micronized dry blend was then sieved with Aerosil 200 Pharma and Natural Orange Flavor WONF or Lemon-Lime Natural Type Flavor using Hand Screen U.S. Std Mesh #30, and the resulting mixture was blended using a 35-liter bin blender to afford a powder formulation for sublingual and/or buccal delivery.

In each formulation method (i.e., fluid bed granulation and dry blending), micronization of the aspirin together with additional excipients (e. g., tri-potassium citrate monohydrate, citric acid anhydrous, aspartame, and sodium lauryl sulfate) was conducted to improve uniform distribution of aspirin in the blend, and the blend uniformity was expected to have been maintained throughout the subsequent process steps.

### Preparation of Pre-Blend Batch Using Air Jet Milling

A micronized aspirin pre-blend was prepared by air jet milling as described above with a batch size of 7.107 kg. To remove large lumps and prevent interference with the feed rate, individual ingredients were screened through US Standard #14 Mesh Hand Screen. Formulation composition and process parameters for preparation of the pre-blend are provided in Table 8 and Table 9, respectively.

TABLE 8
Formulation Composition

| Ingredients | Amount (g) |
|---|---|
| Aspirin USP 80 mesh Powder | 6567.6 |
| Citric Acid anhydrous, fine granular | 33.1 |
| Tri-potassium citrate monohydrate, granular | 9.7 |
| Vitasweet Aspartame 100 mesh | 350.8 |
| powder | |
| Sodium Lauryl Sulfate | 146.3 |

TABLE 9
Process Parameters

| Process Parameters | Data | |
|---|---|---|
| Bin Blender (CML-1200/35 L) RPM | 20 | |
| Blending Duration | 3 | min |
| Powder feed rate/Feeder setting | 499.8/460 | |
| Grind air/Feed air pressure | 50/50 | PSIG |
| Milling duration | 25 | min |

The pre-blend batch was then granulated using either top spray granulation or dry blending.

### Preparation of Fluid Bed Granulation Batch Using Tap Spray Process

A fluid bed granulation batch was prepared using a top-spray process in a Glatt GPCG 3.1 fluid bed dryer with a batch size of 3.5 kg. Sodium lauryl sulfate (SLS) at 0.3% concentration in purified water USP (e. g., 200 mL) was sprayed on micronized aspirin pre-blend (e. g., 3.5 kg) during granulation. The resultant aspirin FB granules, Aerosil 200 Pharma, and Natural Orange Flavor WONF (or alternatively, Lemon-Lime Natural Type Flavor) were then sieved using U.S. Standard #30 Mesh Hand Screen and blended in an amount of 4.9 parts Aerosil 200 and 0.57 parts Flavor to 100 parts aspirin fluid bed granules by weight to obtain finished granular product. Process parameters for fluid bed granulation batch are provided in Table 10.

TABLE 10
Process Parameters

| Process Parameters | Data | |
|---|---|---|
| Product Temperature T₀-T_{end} | 15-25° | C. |
| Air Flow T₀-T_{end} | 20-60 | CFM |
| Spray rate T₀-T_{end} | 5-15 | g/min |
| Bin Blender (CML-10/ 10 L) RPM | 20 | |
| Blending Duration | 3 | min |

### Preparation of Dry Blend Batch

A dry blend batch was prepared by blending a pre-blend batch (2.84 kg) with Aerosil 200 (in an amount of 5.3 parts Aerosil 200 to 100 parts aspirin in the pre-blend by weight) in a CM 1200 bin blender with a batch size of 2.7 kg. Process parameters for dry blend granulation batch are provided in Table 11.

TABLE 11
Process Parameters

| Process Parameters | Data | |
|---|---|---|
| Bin Blender (CM-1200/35 L) RPM | 20 | rpm |
| Blending Duration | 3 | min |

### Aspirin Content Assay

The formulations were analyzed for aspirin content using standard methods known in the art. Samples were collected from each formulation using a 1.0 mm die, and the aspirin content for each batch was analyzed by HPLC.

Standard preparation: Approximately 50 mg USP aspirin was transferred into a 50 mL amber volumetric flask and dissolved in 60/40 0.2% phosphoric acid in USP water/acetonitrile diluent to volume. The resulting solution was mixed well to afford a stock solution, which was diluted to a concentration of 0.1 mg/mL.

Sample preparation: formulation amounts equivalent to 200 mg aspirin were transferred into 100 mL volumetric flasks and dissolved in 50 mL of 0.2% phosphoric acid in 60/40 USP water/acetonitrile diluent to volume. The resulting solution was mixed well to afford a stock solution, which was diluted to a concentration of 0.1 mg/mL. The sample solutions were immediately transferred into HPLC vials and maintained in the HPLC system at 4° C.

HPLC Instrument Details: Waters HPLC; Column: Hypersil GOLD aQ 4.6×250 mm, 5 micron; Column temperature: 30° C.; Sample temperature: 4° C.; Injection volume: 10 μL; Mobile Phase A: 02% phosphoric acid in USP water; Mobile Phase B: 0.2 phosphoric acid in ACN; Mobile phase gradient details: 100% A 0-15 min, 40/60 A/B 15-15.5 min, 100% B 15.5-19 min; Flow rate: 1.0 ml/min; Run time: 19 min; UV Detection Method: PDA, Wavelength: 235 nm.

The aspirin content for each formulation was 90-110% of expected aspirin content.

### Blend Uniformity

Top spray granulation and dry blend formulations were analyzed for blend/content uniformity. In the process, ten blend uniformity (BU) samples were collected using a 1.0 mm dic. Blend samples were prepared at concentrations of approximately 0.1 mg/mL. The samples were analyzed by HPLC using the same methods described above for the aspirin content assay.

The mean blend uniformity for each formulation was 90-110%.

### Example 5. a Phase 1, Single-Dose, Three-Treatment, Three-Period Crossover Bioavailability Study of Two Different Formulations of Oral Transmucosal Acetylsalicylic Acid to Standard Oral Aspirin

### Brief Summary

This example describes a Phase 1 clinical study that evaluated bioavailability of ASA and salicylic acid (SA) following sublingual administration of two different formulations of oral transmucosal ASA (OTASA) powder compared to oral administration of chewable aspirin tablets in healthy subjects.

### Detailed Description

This was a Phase I, single-dose, three-treatment, three-period crossover study of two different formulations of OTASA, 162.5 mg and two chewable ASA tablets, 81 mg in healthy volunteers. Subjects were randomized 1:1:1 to receive treatments A, B, and C in one of three sequences: ABC, CAB, or BCA. The treatment periods were separated by a 14-day washout period.

### Eligibility Criteria:

### Inclusion Criteria:

1. Male or female subjects, 40-65 years of age (inclusive).
2. Body Mass Index (BMI) between 19 and 32 kg/m² (inclusive).
3. Females subjects of childbearing potential must have a negative pregnancy test and use acceptable contraception or abstain from sex during the study.
4. Non-vasectomized male subjects with partners of childbearing potential must use acceptable contraception during the study.
5. Otherwise healthy, with no clinically significant abnormalities based on medical history, physical exam, lab tests, vital signs, and ECG.
6. No alcohol within 24 h before dosing, during the inpatient period, and 24 h before outpatient visits.
7. No prescription medications from 14 days before dosing through study end, unless approved by the investigator and sponsor medical monitor.
8. No OTC medications or herbal products from 14 days before dosing through final discharge, unless approved.
9. Signed informed consent document indicating that they understand the purpose of and procedures required for the study and are willing to participate in the study.
10. Willing/able to adhere to the study visit schedule and other requirements, prohibitions and restrictions specified in this protocol.

### Exclusion Criteria:

1. Pregnant or lactating females.
2. Current or history of any clinically significant illness or disorder deemed exclusionary by the investigator.
3. History of hypersensitivity or allergy to aspirin or other NSAIDs.
4. Phenylketonuria (PKU) or history of allergy to aspartame.
5. Platelet count <150,000 or >450,000 μL.
6. Have an active acute or chronic infection or diagnosed latent infection.
7. Acute illness, including common cold, within 7 days prior to the study or have had major illness or hospitalization within 1 month prior to the study.
8. Major or traumatic surgery within 12 weeks of screening.
9. Any subject who plans to undergo elective surgery within 4 weeks prior to study agent administration and through the end of the study.
10. Positive serology test for human immunodeficiency virus (HIV) antibodies, hepatitis B surface antigen (HBsAg), or hepatitis C virus (HCV) antibodies at screening.
11. Recent history (within previous 6 months) of alcohol or drug abuse.
12. Have smoked tobacco or marijuana or used e-cigarettes, vapes or similar products to inhale active substances within 6 months prior to dosing.
13. Have positive urine toxicology screen at screening or Day −1 for substances of abuse including but not limited to cocaine, cannabinoids, amphetamines, benzodiazepines, barbiturates, opiates and methadone.
14. Have a positive alcohol breath test at screening and/or prior to study agent administration.
15. Consumes, on average, more than approximately 500 mg/day of caffeine (as contained in 5 cups of tea or coffee or 8 cans of soda or other caffeinated products per day).
16. Donated blood or platelets within 60 days prior to screening.
17. Have a history of active drug and/or food allergy or other active allergic disease requiring the constant use of medications, or a history of severe allergic reaction, angioedema or anaphylaxis.
18. Received any other experimental therapy or new investigational agent within 30 days or 5 half-lives (whichever is longer) of study agent administration.

### Study Plan:

Primary Objectives: To evaluate the bioavailability of acetylsalicylic acid (ASA) over 8 hours following sublingual administration of two different formulations of oral transmucosal acetylsalicylic acid (OTASA) compared to oral administration (chewed and swallowed) of uncoated chewable aspirin tablets (Bayer Corporation, Whippany, NJ, USA) in healthy subjects.

Secondary Objectives: To evaluate:
i. Time from dosing until the OTASA formulations dissolve
ii. Pharmacokinetics of SA over 8 hours after dosing
iii. Serum thromboxane B₂ (TxB2) over 8 hours after dosing

Safety Objectives: Safety was assessed by analysis of adverse events.

Methodology: The OTASA products were administered as 186 mg of powder (162.5 mg ASA) under the tongue while the subject was sitting upright. The powder was held under the tongue without swallowing to allow the powder to dissolve in saliva and be absorbed from the oral cavity. The chewable aspirin tablets were administered as two tablets (162 mg of ASA) to be chewed and swallowed.
Treatment A: A single dose of OTASA Batch 1 (ASA 162.5 mg) was administered sublingually and allowed to dissolve;
Treatment B: A single dose of OTASA Batch 2 (ASA 162.5 mg) was administered sublingually and allowed to dissolve;
Treatment C: A single dose of standard uncoated oral aspirin (ASA 162 mg) chewed and swallowed.

Subjects were randomized 1:1:1 to receive treatments A, B, and C in one of three sequences: ABC, CAB, or BCA.

Study Duration: Subjects attended a screening visit, three treatment periods separated by a washout period of at least 14 days, and an end-of-study visit. The maximum duration in the study was approximately six weeks (screening to last subject's last visit).

### Assessments:

Pharmacokinetics (PK): Venous blood samples of 5 mL were collected to measure ASA and SA in plasma and TxB2 in serum prior to administration (baseline, 0) and 2, 5, 10, 15, 20, 30, 40, and 60 minutes and 1.5, 2, 3, 4, 6, and 8 hours post-dose in all subjects.

Pharmacokinetic parameters calculated from ASA or SA concentration versus time profiles included:
AUC_{0-0.5}—area under the plasma concentration-time curve from the time of administration (baseline or time zero) until the 0.5-hour sample (ASA only, not SA)
AU_{C0-1}—area under the plasma concentration-time curve from the time of administration (baseline or time zero) until the 1-hour sample (ASA only, not SA)
AUC₀₋₈—area under the plasma concentration-time curve from the time of administration (baseline or time zero) until the 8-hour sample
AUC_{0-∞}—AUC extrapolated to infinity
Cₘₐₓ—maximum plasma concentration
Tₘₐₓ—time corresponding to Cₘₐₓ
λz—terminal elimination rate constant
T_{1/2}—terminal elimination half-life

Pharmacodynamics (PD): Venous blood samples of 5 mL were collected to measure TxB2 in serum prior to administration (baseline, 0) and 2-, 5-, 10-, 15-, 20-, 30-, 40-, and 60-minutes and 1.5-, 2-, 3-, 4-, 6-, and 8-hours post-dose.

Pharmacodynamic parameters from the individual serum TxB2 versus time profiles included:
AUC_{0-0.5}—area under the serum TxB2 concentration-time curve from the time of administration (baseline or time zero) until the 0.5-hour sample
AUC₀₋₁—area under the serum TxB2 concentration-time curve from the time of administration (baseline or time zero) until the 1-hour sample
AUC₀₋₈—Area under serum TxB2 percent inhibition-time curve from baseline (time zero) until the 8-hour sample
I—% of inhibition from baseline (time zero) at each time point
Iₘₐₓ—Observed maximum percent inhibition
T_{Imax}—Time to maximum percent inhibition
Cₘᵢₙ—Observed minimum up to 8 hours after dosing
T_{Cmin}—Time to minimum up to 8 hours after dosing

Safety: Safety was evaluated by analysis of:
Adverse events from Period 1 through study exit
Physical examinations, vital signs assessments, and clinical laboratory testing at each visit; 12-lead ECG at follow-up/discharge visit.

### Statistical Methods and Analysis:

Pharmacokinetics (PK): ASA and SA plasma concentrations and PK parameters were evaluated by descriptive statistics and presented in tables, figures and listings by treatment.

Pharmacodynamics (PD): Serum TxB2 concentrations and PD parameters were evaluated by descriptive statistics and presented in tables, figures and listings by treatment.

Safety: Safety analyses were evaluated by descriptive statistics and presented in tables and listings by treatment.

### Study Population Characteristics:

The study was conducted from 19 May 2025 to 20 Jun. 2025. One investigator participated in the study. In total, twelve subjects were screened, and six subjects were randomized to three treatment sequences/groups.

Six subjects enrolled and analyzed: 4 male/2 female; 3 white/3 black race; 2 of Hispanic or Latino ethnicity; median height 169.0 cm (range: 156.0-175.5); median weight 79.9 kgs (range: 61.3-89.4); median BMI: 27.5 kg/m2 (range: 25.0-31.7).

All randomized subjects (n=6) completed the study. Subject disposition data are summarized in FIG. 3.

### Results:

### Pharmacokinetic Results

Mean plasma ASA concentrations during the first thirty minutes post-dose (AUC₀₋₃₀) were higher following sublingual administration of OTASA Batch 1 and OTASA Batch 2 compared to the chewed uncoated tablets (unadjusted p=0.046 and p=0.049, respectively, paired test, one-sided). Higher concentrations were observed beginning at 5 minutes post-dose. Mean plasma ASA concentrations vs. time profiles in the first 30 minutes post-dose are presented by treatment in FIG. 4.

No significant differences in plasma ASA concentrations between treatments were observed beyond the first thirty minutes post-dose.

Mean plasma SA (first metabolite of ASA) concentrations during the first thirty minutes after dosing (AUC₀₋₃₀) were also nominally higher following sublingual administration of OTASA Batch 1 and OTASA Batch 2 compared to the chewed uncoated chewable tablets. Higher plasma SA concentrations were observed beginning at 10 minutes post-dose.

The pharmacokinetics of ASA were studied in plasma up to 8 hours after dosing. There were no deviations with respect to sampling times. Mean plasma ASA concentrations vs. time profiles over 8 hours post-dose are presented by treatment in FIG. 5.

Individual ASA plasma PK parameters with descriptive statistics are presented in FIG. 6.

### Pharmacodynamics Results

Mean serum TxB2 concentrations during the first thirty minutes post-dose (AUC₀₋₃₀) were lower following sublingual administration of OTASA Batch 1 and OTASA Batch 2 compared to the chewed uncoated tablets (significant at certain early timepoints, unadjusted p<0.05). Lower concentrations were observed beginning at 2 minutes post-dose.

Concentrations of TxB2 were studied in serum up to 8 hours after dosing. There were no deviations with respect to sampling times. Mean serum TxB2 concentrations vs. time profiles over 8 hours post-dose are presented in FIG. 7.

Mean serum TxB2 concentrations during the first 30 minutes post-dose are presented by treatment in FIG. 8. Mean serum TxB2 inhibition (%) during the first 30 minutes post-dose are presented by treatment in FIG. 9.

Individual serum TxB2 PD parameters with descriptive statistics are presented in FIG. 10.

### SA Plasma Concentration Results

The pharmacokinetics of SA were studied in plasma up to 8 hours after dosing. There were no deviations with respect to sampling times. Mean plasma SA concentrations vs. time profiles over 8 hours post-dose are presented by treatment in FIG. 11. Mean plasma SA concentrations during the first 30 minutes post-dose are presented by treatment in FIG. 12.

Individual SA plasma PK parameters with descriptive statistics are presented in FIG. 13.

### Dissolution Results

Individual times from dosing until each OTASA powder completely dissolved are listed in FIG. 14. Longer oral dissolution times were typically associated with higher plasma ASA Tₘₐₓ and lower plasma ASA AUC₀₋₃₀.

### Safety Results

No adverse events or clinically significant laboratory results, physical examination findings, vital signs or ECGs were reported.

### Additional Results:

To assess early plasma concentrations of ASA and SA, blood samples for PK analysis were collected at frequent intervals during the first hour after dosing and at additional time points up to 8 hours post-dose. For each treatment group, plasma concentration-time data are presented in separate tables for the first hour and for 1.5-8 hours post-dose, followed by a summary table of key PK parameters (Tables 12-14 for Treatment A, Tables 15-17 for Treatment B, and Tables 18-20 for Treatment C; Tables 21-29 follow the same structure for the SA).

TABLE 12
Treatment A Plasma ASA Concentrations over Time (Frist Hour Post-Dosing)

| Subject | 0 | 0.033 | 0.083 | 0.167 | 0.25 | 0.333 | 0.5 | 0.667 | 1 |
|---|---|---|---|---|---|---|---|---|---|
| S1 | 0.00 | 0.00 | 96.46 | 743.72 | 1586.14 | 1251.11 | 1299.27 | 1497.32 | 1007.09 |
| S2 | 0.00 | 86.15 | 1043.30 | 2799.19 | 3199.74 | 2434.09 | 2059.68 | 1475.90 | 1135.91 |
| S3 | 0.00 | 94.60 | 608.03 | 1890.93 | 3232.16 | 3821.73 | 4494.82 | 3199.23 | 1318.25 |
| S4 | 0.00 | 0.00 | 0.00 | 295.52 | 931.50 | 1141.61 | 1255.67 | 1013.49 | 755.35 |
| S5 | 0.00 | 0.00 | 521.23 | 2847.42 | 2900.14 | 3491.95 | 2571.14 | 1938.25 | 740.19 |
| S6 | 0.00 | 0.00 | 782.73 | 2264.29 | 3170.96 | 2821.40 | 1822.33 | 1221.13 | 598.65 |
| MEAN | — | 30.13 | 508.63 | 1,806.85 | 2,503.44 | 2,493.65 | 2,250.49 | 1,724.22 | 925.91 |

TABLE 13
Treatment A Plasma ASA Concentrations
over Time (1.5 to 8 Hours Post-Dosing)

| Subject | 1.5 | 2 | 3 | 4 | 6 | 8 |
|---|---|---|---|---|---|---|
| S1 | 409.61 | 222.58 | 0.00 | 0.00 | 0.00 | 0.00 |
| S2 | 802.47 | 655.66 | 128.53 | 0.00 | 0.00 | 0.00 |
| S3 | 901.26 | 258.11 | 0.00 | 0.00 | 0.00 | 0.00 |
| S4 | 385.56 | 200.77 | 0.00 | 0.00 | 0.00 | 0.00 |
| S5 | 233.82 | 96.79 | 0.00 | 0.00 | 0.00 | 0.00 |
| S6 | 229.62 | 107.15 | 0.00 | 0.00 | 0.00 | 0.00 |
| MEAN | 493.72 | 256.84 | 21.42 | — | — | — |

TABLE 14
Treatment A Plasma ASA Summary PK Parameters

| Subject | Cₘₐₓ | Tₘₐₓ | AUC₀₋₃₀ | AUC0-60 | AUC₀₋₈ h |
|---|---|---|---|---|---|
| S1 | 1,586.14 | 0.25 | 465.25 | 1115.70 | 1739.21 |
| S2 | 3,199.74 | 0.25 | 1048.96 | 1778.89 | 3084.38 |
| S3 | 4,494.82 | 0.50 | 1323.69 | 2717.77 | 3691.54 |
| S4 | 1,255.67 | 0.50 | 349.59 | 833.50 | 1365.69 |
| S5 | 3,491.95 | 0.33 | 1164.47 | 1986.66 | 2361.21 |
| S6 | 3,170.96 | 0.25 | 1009.66 | 1566.57 | 1911.41 |
| MEAN | 2,866.55 | 0.35 | 893.60 | 1,666.51 | 2,358.91 |

TABLE 15
Treatment B Plasma ASA Concentrations over Time (Frist Hour Post-Dosing)

| Subject | 0 | 0.033 | 0.083 | 0.167 | 0.25 | 0.333 | 0.5 | 0.667 | 1 |
|---|---|---|---|---|---|---|---|---|---|
| S1 | 0.00 | 0.00 | 198.52 | 696.28 | 1059.11 | 1169.66 | 1303.04 | 1234.00 | 1054.30 |
| S2 | 0.00 | 0.00 | 589.58 | 3903.03 | 5463.07 | 4962.84 | 3518.12 | 1650.04 | 831.14 |
| S3 | 0.00 | 119.43 | 875.94 | 1824.82 | 2258.52 | 2851.06 | 3283.04 | 3414.54 | 2120.69 |
| S4 | 0.00 | 0.00 | 0.00 | 438.66 | 688.85 | 849.59 | 1149.34 | 1130.49 | 772.07 |
| S5 | 0.00 | 0.00 | 269.68 | 2783.13 | 2835.64 | 3878.66 | 2892.27 | 1354.96 | 873.70 |
| S6 | 0.00 | 0.00 | 2577.85 | 2864.81 | 6075.52 | 3592.30 | 2123.70 | 1451.70 | 651.03 |
| MEAN | | 19.91 | 751.93 | 2,085.12 | 3,063.45 | 2,884.02 | 2,378.25 | 1,705.96 | 1,050.49 |

TABLE 16
Treatment B Plasma ASA Concentrations
over Time (1.5 to 8 Hours Post-Dosing)

| Subject | 1.5 | 2 | 3 | 4 | 6 | 8 |
|---|---|---|---|---|---|---|
| S1 | 628.67 | 333.84 | 58.31 | 0.00 | 0.00 | 0.00 |
| S2 | 341.49 | 140.62 | 0.00 | 0.00 | 0.00 | 0.00 |
| S3 | 738.07 | 214.11 | 0.00 | 0.00 | 0.00 | 0.00 |
| S4 | 447.05 | 200.48 | 0.00 | 0.00 | 0.00 | 0.00 |
| S5 | 347.38 | 139.06 | 0.00 | 0.00 | 0.00 | 0.00 |
| S6 | 147.55 | 77.99 | 0.00 | 0.00 | 0.00 | 0.00 |
| MEAN | 441.70 | 184.35 | 9.72 | — | — | — |

TABLE 17
Treatment B Plasma ASA Summary PK Parameters

| Subject | Cₘₐₓ | Tₘₐₓ | AUC₀₋₃₀ | AUC0-60 | AUC₀₋₈ h |
|---|---|---|---|---|---|
| S1 | 1,303.04 | 0.50 | 414.31 | 1007.11 | 1893.71 |
| S2 | 5,463.07 | 0.25 | 1733.35 | 2577.56 | 3061.55 |
| S3 | 3,414.54 | 0.67 | 1033.62 | 2514.29 | 3574.08 |
| S4 | 1,149.34 | 0.50 | 295.94 | 803.02 | 1369.92 |
| S5 | 3,878.66 | 0.33 | 1212.06 | 1937.44 | 2433.85 |
| S6 | 6,075.52 | 0.25 | 1542.90 | 2191.30 | 2486.33 |
| MEAN | 3,547.36 | 0.42 | 1,038.70 | 1,838.45 | 2,469.91 |

TABLE 18
Treatment C Plasma ASA Concentrations over Time (Frist Hour Post-Dosing)

| Subject | 0 | 0.033 | 0.083 | 0.167 | 0.25 | 0.333 | 0.5 | 0.667 | 1 |
|---|---|---|---|---|---|---|---|---|---|
| S1 | 0.00 | 0.00 | 328.74 | 866.92 | 2352.63 | 2217.21 | 1903.58 | 1893.54 | 949.33 |
| S2 | 0.00 | 109.66 | 369.92 | 841.63 | 1254.10 | 1766.27 | 3538.28 | 2340.61 | 1153.83 |
| S3 | 0.00 | 0.00 | 335.29 | 1197.39 | 2066.30 | 2393.44 | 1923.14 | 1938.99 | 1254.37 |
| S4 | 0.00 | 59.16 | 0.00 | 239.83 | 492.68 | 754.63 | 875.42 | 1084.35 | 942.91 |
| S5 | 0.00 | 0.00 | 132.78 | 647.89 | 1208.56 | 1855.61 | 2681.68 | 2053.83 | 945.30 |
| S6 | 0.00 | 0.00 | 228.26 | 634.89 | 1555.12 | 2438.15 | 2846.98 | 2139.61 | 1193.16 |
| MEAN | — | 28.14 | 232.50 | 738.09 | 1,488.23 | 1,904.22 | 2,294.85 | 1,908.49 | 1,073.15 |

TABLE 19
Treatment C Plasma ASA Concentrations
over Time (1.5 to 8 Hours Post-Dosing)

| Subject | 1.5 | 2 | 3 | 4 | 6 | 8 |
|---|---|---|---|---|---|---|
| S1 | 360.90 | 123.53 | 0.00 | 0.00 | 0.00 | 0.00 |
| S2 | 520.21 | 192.27 | 0.00 | 0.00 | 0.00 | 0.00 |
| S3 | 758.92 | 311.45 | 0.00 | 0.00 | 0.00 | 0.00 |
| S4 | 598.34 | 246.88 | 63.60 | 0.00 | 0.00 | 0.00 |
| S5 | 334.54 | 127.58 | 0.00 | 0.00 | 0.00 | 0.00 |
| S6 | 361.32 | 156.74 | 0.00 | 0.00 | 0.00 | 0.00 |
| MEAN | 489.04 | 193.08 | 10.60 | — | — | — |

TABLE 20
Treatment C Plasma ASA Summary PK Parameters

| Subject | Cₘₐₓ | Tₘₐₓ | AUC₀₋₃₀ | AUC0-60 | AUC₀₋₈ h |
|---|---|---|---|---|---|
| S1 | 2,352.63 | 0.25 | 725.99 | 1516.23 | 2026.66 |
| S2 | 3,538.28 | 0.50 | 719.52 | 1791.83 | 2484.59 |
| S3 | 2,393.44 | 0.33 | 753.77 | 1607.84 | 2534.48 |
| S4 | 1,084.35 | 0.67 | 230.79 | 731.98 | 1515.64 |
| S5 | 2,681.68 | 0.50 | 618.98 | 1513.46 | 2012.74 |
| S6 | 2,846.98 | 0.50 | 739.74 | 1710.75 | 2307.25 |
| MEAN | 2,482.89 | 0.46 | 631.46 | 1,478.68 | 2,146.89 |

TABLE 21
Treatment A Plasma SA Concentrations over Time (Frist Hour Post-Dosing)

| Subject | 0 | 0.033 | 0.083 | 0.167 | 0.25 | 0.333 | 0.5 | 0.667 | 1 |
|---|---|---|---|---|---|---|---|---|---|
| S1 | 0.00 | 0.00 | 0.00 | 511.61 | 1838.08 | 2785.21 | 4363.92 | 6155.73 | 7248.08 |
| S2 | 0.00 | 0.00 | 577.21 | 3521.28 | 5205.28 | 5799.33 | 6902.77 | 6842.17 | 7903.55 |
| S3 | 0.00 | 0.00 | 0.00 | 732.06 | 2243.91 | 4018.49 | 9133.67 | 10259.18 | 9238.27 |
| S4 | 0.00 | 0.00 | 0.00 | 0.00 | 775.19 | 1678.95 | 3279.76 | 4350.03 | 5502.29 |
| S5 | 0.00 | 0.00 | 0.00 | 1771.48 | 2973.99 | 5352.56 | 7567.53 | 9025.64 | 9327.83 |
| S6 | 0.00 | 0.00 | 287.10 | 2014.97 | 4262.25 | 5119.63 | 6634.77 | 6409.64 | 6075.26 |
| MEAN | — | — | 144.05 | 1,425.23 | 2,883.12 | 4,125.70 | 6,313.74 | 7,173.73 | 7,549.21 |

TABLE 22
Treatment A Plasma SA Concentrations over Time (1.5 to 8 Hours Post-Dosing)

| Subject | 1.5 | 2 | 3 | 4 | 6 | 8 |
|---|---|---|---|---|---|---|
| S1 | 8190.33 | 8052.37 | 6631.63 | 4991.80 | 2647.03 | 1389.36 |
| S2 | 9434.27 | 9512.19 | 8552.04 | 7771.33 | 5190.31 | 3807.19 |
| S3 | 9224.07 | 7895.53 | 6739.77 | 4766.92 | 2365.65 | 1160.26 |
| S4 | 6250.36 | 5739.49 | 4171.45 | 3380.36 | 1712.32 | 1035.24 |
| S5 | 8331.50 | 7784.41 | 5516.89 | 4476.65 | 2416.62 | 1360.97 |
| S6 | 6755.36 | 6215.30 | 4042.28 | 2906.17 | 1690.88 | 878.40 |
| MEAN | 8,030.98 | 7,533.22 | 5,942.34 | 4,715.54 | 2,670.47 | 1,605.24 |

TABLE 23
Treatment A Plasma SA Summary PK Parameters

| Subject | Cₘₐₓ | Tₘₐₓ | AUC₀₋₃₀ | AUC0-60 | AUC₀₋₈ h |
|---|---|---|---|---|---|
| S1 | 8,190.33 | 1.50 | 907.62 | 4018.22 | 36767.44 |
| S2 | 9,512.19 | 2.00 | 2065.84 | 5668.87 | 53892.88 |
| S3 | 10,259.18 | 0.67 | 1511.45 | 6377.09 | 39002.05 |
| S4 | 6,250.36 | 1.50 | 547.78 | 2825.65 | 25332.89 |
| S5 | 9,327.83 | 1.00 | 1695.15 | 6136.83 | 36898.92 |
| S6 | 6,755.36 | 1.50 | 1735.09 | 4902.94 | 27122.61 |
| MEAN | 8,382.54 | 1.36 | 1,410.49 | 4,988.27 | 36,502.80 |

TABLE 24
Treatment B Plasma SA Concentrations over Time (Frist Hour Post-Dosing)

| Subject | 0 | 0.033 | 0.083 | 0.167 | 0.25 | 0.333 | 0.5 | 0.667 | 1 |
|---|---|---|---|---|---|---|---|---|---|
| S1 | 0.00 | 0.00 | 0.00 | 695.25 | 1812.59 | 2415.07 | 3778.32 | 4945.30 | 6694.42 |
| S2 | 0.00 | 0.00 | 239.34 | 3777.76 | 8651.52 | 10666.17 | 10627.14 | 10904.92 | 11100.33 |
| S3 | 0.00 | 0.00 | 247.23 | 745.03 | 1444.40 | 2781.81 | 5264.00 | 7857.17 | 10775.92 |
| S4 | 318.11 | 332.33 | 279.83 | 483.35 | 1152.56 | 1619.47 | 3252.55 | 4393.02 | 5463.75 |
| S5 | 0.00 | 0.00 | 0.00 | 3226.31 | 4672.33 | 6774.74 | 6977.75 | 7314.19 | 8057.61 |
| S6 | 0.00 | 0.00 | 745.60 | 2170.30 | 6243.51 | 6147.43 | 6818.25 | 7971.82 | 8192.02 |
| MEAN | 53.02 | 55.39 | 252.00 | 1,849.67 | 3,996.15 | 5,067.45 | 6,119.67 | 7,231.07 | 8,380.68 |

TABLE 25
Treatment B Plasma SA Concentrations over Time (1.5 to 8 Hours Post-Dosing)

| Subject | 1.5 | 2 | 3 | 4 | 6 | 8 |
|---|---|---|---|---|---|---|
| S1 | 7912.51 | 6586.02 | 5835.19 | 4499.75 | 2496.40 | 1327.63 |
| S2 | 10039.42 | 9675.05 | 7875.92 | 6974.43 | 5237.44 | 4051.83 |
| S3 | 9984.48 | 8753.63 | 6156.80 | 5057.58 | 2704.63 | 1165.76 |
| S4 | 6477.78 | 6078.30 | 5015.40 | 4068.92 | 2320.85 | 1386.85 |
| S5 | 7955.73 | 6755.92 | 5228.57 | 4260.05 | 2531.41 | 1241.70 |
| S6 | 6503.96 | 6085.84 | 4765.45 | 3801.65 | 1875.62 | 837.65 |
| MEAN | 8,145.65 | 7,322.46 | 5,812.89 | 4,777.06 | 2,861.06 | 1,668.57 |

TABLE 26
Treatment B Plasma SA Summary PK Parameters

| Subject | Cₘₐₓ | Tₘₐₓ | AUC₀₋₃₀ | AUC0-60 | AUC₀₋₈ h |
|---|---|---|---|---|---|
| S1 | 7,912.51 | 1.50 | 825.73 | 3492.65 | 32967.27 |
| S2 | 11,100.33 | 1.00 | 3270.60 | 8732.48 | 56647.83 |
| S3 | 10,775.92 | 1.00 | 985.33 | 5184.27 | 39753.91 |
| S4 | 6,477.78 | 1.50 | 647.61 | 2927.54 | 29238.42 |
| S5 | 8,057.61 | 1.00 | 2086.54 | 5839.50 | 34821.88 |
| S6 | 8,192.02 | 1.00 | 2087.47 | 6013.95 | 30935.13 |
| MEAN | 8,752.70 | 1.17 | 1,650.55 | 5,365.07 | 37,394.07 |

TABLE 27
Treatment C Plasma SA Concentrations over Time (Frist Hour Post-Dosing)

| Subject | 0 | 0.033 | 0.083 | 0.167 | 0.25 | 0.333 | 0.5 | 0.667 | 1 |
|---|---|---|---|---|---|---|---|---|---|
| S1 | 0.00 | 0.00 | 0.00 | 966.62 | 3001.71 | 4211.30 | 6038.68 | 7610.58 | 8563.02 |
| S2 | 0.00 | 0.00 | 481.10 | 1655.17 | 2554.30 | 3844.39 | 7870.01 | 8604.19 | 10032.04 |
| S3 | 0.00 | 0.00 | 259.88 | 1833.63 | 3781.28 | 5283.65 | 5602.85 | 7130.42 | 8275.98 |
| S4 | 0.00 | 0.00 | 0.00 | 223.29 | 569.07 | 1442.94 | 3007.77 | 4115.84 | 5423.88 |
| S5 | 0.00 | 0.00 | 0.00 | 567.45 | 1693.76 | 3329.36 | 6082.10 | 7652.76 | 8618.42 |
| S6 | 0.00 | 0.00 | 0.00 | 405.96 | 1129.90 | 2698.67 | 5431.60 | 6245.83 | 7293.13 |
| MEAN | — | — | 123.50 | 942.02 | 2,121.67 | 3,468.39 | 5,672.17 | 6,893.27 | 8,034.41 |

TABLE 28
Treatment C Plasma SA Concentrations over Time (1.5 to 8 Hours Post-Dosing)

| Subject | 1.5 | 2 | 3 | 4 | 6 | 8 |
|---|---|---|---|---|---|---|
| S1 | 7898.87 | 7428.05 | 5684.53 | 3650.39 | 1902.99 | 1241.41 |
| S2 | 10593.04 | 9738.13 | 7996.68 | 6681.51 | 4617.96 | 3706.93 |
| S3 | 8491.47 | 9222.57 | 6807.79 | 5077.40 | 2684.98 | 1485.02 |
| S4 | 6788.87 | 5916.59 | 5204.99 | 3841.20 | 2296.38 | 1050.10 |
| S5 | 7949.16 | 7558.08 | 5788.25 | 4586.00 | 2730.93 | 1287.00 |
| S6 | 6805.96 | 5894.75 | 4246.92 | 3101.46 | 1498.57 | 859.94 |
| MEAN | 8,087.90 | 7,626.36 | 5,954.86 | 4,489.66 | 2,621.97 | 1,605.07 |

TABLE 29
Treatment C Plasma SA Summary PK Parameters

| Subject | Cₘₐₓ | Tₘₐₓ | AUC₀₋₃₀ | AUC0-60 | AUC₀₋₈ h |
|---|---|---|---|---|---|
| S1 | 8,563.02 | 1.00 | 1360.33 | 5193.37 | 33062.10 |
| S2 | 10,593.04 | 1.50 | 1519.25 | 5998.13 | 52068.06 |
| S3 | 9,222.57 | 2.00 | 1612.59 | 5241.43 | 39751.96 |
| S4 | 6,788.87 | 1.50 | 497.05 | 2680.63 | 28478.13 |
| S5 | 8,618.42 | 1.00 | 1111.45 | 4967.88 | 36181.74 |
| S6 | 7,293.13 | 1.00 | 917.96 | 4147.57 | 26551.08 |
| MEAN | 8,513.18 | 1.33 | 1,169.77 | 4,704.84 | 36,015.51 |

As shown in Table 6-14, plasma concentrations of ASA rose more rapidly following administration of Treatments A and B than with Treatment C. Within the first 30 minutes post-dose (0.083, 0.167, 0.25, and 0.333 hours), mean plasma concentrations of ASA for Treatments A and B were substantially higher than those observed for Treatment C. By 0.5 hours post-dose, plasma concentrations for Treatment C increased to levels comparable to A and B. Consistent with this faster initial absorption, the mean Cₘₐₓ values for Treatments A and B were also higher than that of Treatment C.

Table 15-23 indicate that for SA, Treatments A and B likewise produced more rapid systemic exposure than Treatment C. Between 0.083 and 0.667 hours post-dose (0.083, 0.167, 0.25, 0.333, 0.5, and 0.667 hours), mean plasma concentrations of SA were much higher for Treatments A and B relative to Treatment C, which reached comparable concentrations only at 1 hour post-dose. These findings indicate that both Treatments A and B achieve faster absorption and earlier systemic availability of ASA and SA than the control formulation (Treatment C).

### CONCLUSION

The OTASA formulations evaluated in this clinical trial are investigational new drugs proposed for treatment of suspected acute myocardial infarction with the objective of reducing the time to systemic bioavailability and pharmacodynamic effect of ASA.

In this proof-of-concept, single-dose, three-treatment, three-period, crossover bioavailability study, sublingual administration of each OTASA powder formulation, Batch 1 and Batch 2, was associated with:
more rapid absorption evidenced by higher plasma ASA concentrations (AUC₀₋₃₀) during the first thirty minutes post-dose (unadjusted p=0.046 and p=0.049, respectively, paired t test, one-sided),
more rapid pharmacodynamic effect measured by inhibition of serum TxB2 during the first 30 minutes after dosing (significant at certain early timepoints, unadjusted p<0.05, paired t test, one-sided), and
nominally higher plasma SA concentrations during the first thirty minutes after dosing compared to the chewed uncoated chewable aspirin tablets.

Times from administration of the OTASA powder formulations to complete dissolution of the powder in the oral cavity as reported by subjects and confirmed by the clinical staff were nominally longer for OTASA Batch 1 than for Batch 1 (mean=3.00 minutes vs. 2.10 minutes, not significant). Longer oral dissolution times were typically associated with longer times to maximum plasma ASA concentrations (Tₘₐₓ) and lower plasma ASA concentrations during the first thirty minutes post-dose (AUC₀₋₃₀).

OTASA Batch 1, manufactured by a fluid bed granulation process, did not show any apparent advantage in time to oral dissolution, bioavailability, or pharmacodynamic effect compared to OTASA Batch 2, and therefore, the added processing step seems unwarranted. Sublingual administration of a single dose of 162.5 mg ASA as OTASA powder Batch 1 or OTASA powder Batch 2 and oral administration of a single dose of 162 mg ASA as chewed and swallowed uncoated chewable aspirin tablets were safe and well tolerated in healthy subjects 40 to 65 years of age.

The results of this clinical trial support further development of sublingual OTASA powder, 162.5 mg for treatment of suspected acute myocardial infarction with the objective of achieving more rapid inhibition of platelet aggregation than current standard of care.

### Example 6. A Clinical Study of Two Different Formulations of Oral Transmucosal Acetylsalicylic Acid in the Treatment of Myocardial Infarction (MI)

### Brief Summary:

This example describes a clinical study of ASA in the treatment of myocardial infarction (MI). ASA is expected to achieve rapid absorption and exert potent antiplatelet effects, which may help restore coronary blood flow, limit infarct size, and reduce the risk of subsequent ischemic events. By inhibiting platelet aggregation early after onset, ASA has the potential to improve myocardial reperfusion, enhance recovery of cardiac function, and lower the incidence of recurrent thrombosis.

This study is intended to assess the pharmacokinetics, pharmacodynamics, efficacy, and safety of ASA. The study will evaluate the absorption rate and antiplatelet effects of sublingual administration of two different formulations of OTASA powder compared to oral administration of chewable aspirin tablets in subjects with MI.

### Eligibility Criteria:

### Inclusion Criteria:

1. Male or female subjects, 40-65 years of age (inclusive).
2. Body Mass Index (BMI) between 19 and 32 kg/m² (inclusive).
3. Confirmed diagnosis of MI according to ECG results, imagining or other relevant tests.
4. Females subjects of childbearing potential must have a negative pregnancy test and use acceptable contraception or abstain from sex during the study.
5. Non-vasectomized male subjects with partners of childbearing potential must use acceptable contraception during the study.
6. No alcohol within 24 h before dosing, during the inpatient period, and 24 h before outpatient visits.
7. No prescription medications from 14 days before dosing through study end, unless approved by the investigator and sponsor medical monitor.
8. No OTC medications or herbal products from 14 days before dosing through final discharge, unless approved.

### Exclusion Criteria:

1. Pregnant or lactating females.
2. Current or history of any clinically significant illness or disorder deemed exclusionary by the investigator.
3. History of hypersensitivity or allergy to aspirin or other NSAIDs.
4. Phenylketonuria (PKU) or history of allergy to aspartame.
5. Platelet count <150,000 or >450,000 μL.
6. Have an active acute or chronic infection or diagnosed latent infection.
7. Acute illness, including common cold, within 7 days prior to the study or have had major illness or hospitalization within 1 month prior to the study.
8. Major or traumatic surgery within 12 weeks of screening

### Study Plan:

Primary Objectives: To evaluate efficacy, pharmacokinetics and pharmacodynamics of ASA in the treatment of MI following sublingual administration of two different formulations of OTASA compared to oral administration of chewable aspirin tablets in healthy subjects.

Secondary Objectives: To assess safety and tolerability of ASA in treatment of MI.

### Methodology:

This is a randomized, double-blind study. The OTASA products is administered as 186 mg of powder (162.5 mg ASA) under the tongue while the subject is sitting upright. The powder is held under the tongue without swallowing to allow the powder to dissolve in saliva and be absorbed from the oral cavity. The chewable aspirin tablets are administered as two tablets (162 mg of ASA) to be chewed and swallowed.

There are three arms in this study:
Arm 1: Subjects with MI will be given once daily a single dose of OTASA Batch 1 (ASA 162.5 mg) administered sublingually and allowed to dissolve;
Arm 2: Subjects with MI will be given once daily a single dose of OTASA Batch 2 (ASA 162.5 mg) administered sublingually and allowed to dissolve;
Arm 3: Subjects with MI will be given once daily a single dose of standard oral aspirin (ASA 162 mg) chewed and swallowed.

### Study Duration:

Total participation per subject: approximately 16 weeks.

Treatment period: 28 days.

Follow-up: 30 days after last dose.

### Assessments:

Venous blood samples will be collected to measure ASA and SA in plasma and TxB2 in serum prior to administration (baseline, 0) and 2, 5, 10, 15, 20, 30, 40, and 60 minutes and 1.5, 2, 3, 4, 6, and 8 hours post-dose in all subjects.

Pharmacokinetic parameters from ASA or SA concentration versus time profiles:
AUC_{0-0.5}—area under the plasma concentration-time curve from the time of administration (baseline or time zero) until the 0.5-hour sample (ASA only, not SA)
AUC₀₋₁—area under the plasma concentration-time curve from the time of administration (baseline or time zero) until the 1-hour sample (ASA only, not SA)
AUC₀₋₈-area under the plasma concentration-time curve from the time of administration (baseline or time zero) until the 8-hour sample
AUC_{0-∞}—AUC extrapolated to infinity
Cₘₐₓ—maximum plasma concentration
Tₘₐₓ—time corresponding to Cₘₐₓ
λz—terminal elimination rate constant
T_{1/2}—terminal elimination half-life

Pharmacodynamic parameters from the individual serum TxB2 versus time profiles:
I—% of inhibition from baseline (time zero) at each time point
Iₘₐₓ—Observed maximum percent inhibition
Tₘₐₓ—Time to maximum percent inhibition
AUC₀₋₈—Area under percent inhibition taken directly from the inhibition time course profile from baseline (time zero) until the 8-hour sample
Cₘᵢₙ—Observed minimum up to 8 hours after dosing
Tₘᵢₙ—Time to minimum up to 8 hours after dosing
AUC₀₋₈—Area under the concentration time curve from baseline (time zero) until the 8-hour sample

### Efficacy Assessments:

Efficacy will be assessed by investigator evaluation of myocardial reperfusion and prevention of recurrent ischemic events. ECGs will be performed at baseline, shortly after dosing, and at scheduled time points to assess ST-segment resolution. Biomarkers such as myoglobin and CK-MB will be measured to monitor myocardial injury. Echocardiography may be performed as needed to assess cardiac function.

### Safety Assessment:

Safety is monitored through the collection of adverse events, vital signs, physical examinations, and routine laboratory tests (hematology, blood chemistry, and urinalysis). ECGs are performed at baseline and scheduled intervals to detect any cardiac rhythm abnormalities. Serious adverse events are documented and reported according to regulatory requirements.

1. A pharmaceutical formulation suitable for oral mucosal (e.g., sublingual and/or buccal) administration comprising:
91 mg to 200 mg micronized aspirin;
a buffer;
a surfactant; and
one or more pharmaceutically acceptable excipients,
wherein the pH of a solution of the formulation dissolved in 1 mL distilled water is less than or equal to 3.

2. The pharmaceutical formulation of claim 1, wherein the formulation comprises granules comprising an intragranular fraction, wherein the intragranular fraction comprises the micronized aspirin.

3. The pharmaceutical formulation of claim 2, wherein the intragranular fraction further comprises the buffer.

4. The pharmaceutical formulation of claim 1, wherein the formulation does not comprise granules.

5. The pharmaceutical formulation of claim 1, wherein the pH of a solution of the formulation dissolved in 1 mL distilled water is less than 2.8.

6. The pharmaceutical formulation of claim 1, wherein the pH of a solution of the formulation dissolved in 1 ml distilled water is less than 2.5.

7. The pharmaceutical formulation of claim 1, wherein the pH of a solution of the formulation dissolved in 1 ml distilled water is less than 2.2.

8. The pharmaceutical formulation of claim 1, wherein the pH of a solution of the formulation dissolved in 1 ml distilled water is 2 to 2.5.

9. The pharmaceutical formulation of claim 1, wherein the pH of a solution of the formulation dissolved in 1 ml distilled water is 1.8 to 2.5.

10. The pharmaceutical formulation of claim 1, wherein the buffer concentration of the formulation is equal to or greater than 50 mM when dissolved in 1 mL of distilled water.

11. The pharmaceutical formulation of claim 1, wherein the formulation comprises 100 mg to 163 mg micronized aspirin.

12. The pharmaceutical formulation of claim 1, wherein the formulation comprises 145 mg to 163 mg micronized aspirin.

13. (canceled)

14. The pharmaceutical formulation of claim 1, wherein the buffer comprises citric acid, or a hydrate thereof, and a citrate salt, or a hydrate thereof.

15. The pharmaceutical formulation of claim 1, wherein the buffer consists of citric acid, or a hydrate thereof, and a citrate salt, or a hydrate thereof.

16. The pharmaceutical formulation of claim 14, wherein the citric acid or hydrate thereof is citric acid anhydrous.

17. The pharmaceutical formulation of claim 14, wherein the citrate salt or hydrate thereof is tri-potassium citrate monohydrate.

18. The pharmaceutical formulation of claim 1, wherein the surfactant is an anionic surfactant.

19. The pharmaceutical formulation of claim 1, wherein the surfactant is selected from the group consisting of sodium lauryl sulfate, ammonium lauryl sulfate, potassium lauryl sulfonate, potassium lauryl phosphate, sodium deoxycholate, and potassium deoxycholate.

20. The pharmaceutical formulation of claim 1, wherein the surfactant is sodium lauryl sulfate.

21-46. (canceled)

47. A pharmaceutical formulation suitable for oral mucosal (e.g., sublingual and/or buccal) administration comprising:
162.5 mg micronized aspirin;
0.82 mg citric acid anhydrous;
0.24 mg tri-potassium citrate monohydrate;
3.65 mg sodium lauryl sulfate; and
one or more pharmaceutically acceptable excipients.

48. The pharmaceutical formulation of claim 47, wherein the formulation comprises an anti-settling and/or anti-sagging agent.

49. The pharmaceutical formulation of claim 48, wherein the anti-settling and/or anti-sagging agent is fumed silica.

50-51. (canceled)

52. The pharmaceutical formulation of claim 47, wherein the formulation comprises a sweetening agent.

53. The pharmaceutical formulation of claim 52, wherein the sweetening agent is aspartame.

54-58. (canceled)

59. The pharmaceutical formulation of claim 1, wherein the formulation is a solid dosage form.

60. The pharmaceutical formulation of claim 59, wherein the solid dosage form is an orally disintegrating tablet.

61. The pharmaceutical formulation of claim 59, wherein the solid dosage form is a powder.

62. (canceled)

63. A method of treating a disease, disorder, or condition in a subject in need thereof, the method comprising administering to the subject one or more pharmaceutical formulations of claim 1.

64. The method of claim 63, wherein the disease, disorder, or condition is selected from pain, fever, and swelling.

65. The method of claim 63, wherein the disease, disorder, or condition is myocardial infarction.

66-67. (canceled)
